(19) United States
(12) Patent Application Publication
(10) Pub. No.: US 2013/0059774 A1
(43) Pub. Date: Mar. 7, 2013

(54) EFFLUX PUMP INHIBITORS

(71) Applicants: Mahesh Vithalbhai Patel, Aurangabad (IN); Sachin Subhash Bhagwat, Aurangabad (IN); Mohammad Alam Jafri, Aligarh (IN)

(72) Inventors: Mahesh Vithalbhai Patel, Aurangabad (IN); Sachin Subhash Bhagwat, Aurangabad (IN); Mohammad Alam Jafri, Aligarh (IN)

(21) Appl. No.: 13/578,428

(22) PCT Filed: Apr. 1, 2010
(86) PCT No.: PCT/IB2010/051434

§ 371 Date: Oct. 30, 2012

Related U.S. Application Data
(60) Provisional application No. 60/222,201, filed on Aug. 1, 2000.
(60) Provisional application No. 60/286,291, filed on Apr. 25, 2001.

(30) Foreign Application Priority Data
Aug. 19, 2000 (US) 09640947
May 7, 2001 (US) 09850669

(51) Int. Cl. A61K 31/546; A61K 31/7048; A61K 31/65; A61P 31/00; C12N 1/10; A61P 31/10; C12N 1/20; C12N 1/14; C12N 1/16; A61K 38/12; A61P 31/04

(52) U.S. Cl. 514/2.9; 514/203; 514/2.3; 514/29; 514/3.6; 514/154; 435/252.1; 435/254.1; 435/255.1; 435/258.1

(57) ABSTRACT

Novel compositions and methods of reducing microbial resistance to antimicrobial agents and treating infections are disclosed. In particular, compositions and methods of inhibiting efflux pump activity, treating infection and methods of enhancing antimicrobial activity of antimicrobial agents are provided.

### RELATED PATENT APPLICATIONS

This application claims the benefit of Indian Provisional Patent Application Nos. 424/MUM/2010 filed Feb. 16, 2010 and 425/MUM/2010 filed Feb. 16, 2010, the disclosures of which are incorporated herein by reference in their entireties as if fully rewritten herein.

### FIELD OF THE INVENTION

This invention relates to the field of antimicrobial agents and to the use of β-lactam compounds and analogous compositions as efflux pump inhibitors and/or porin modulators, which may be administered with antimicrobial agents for the treatment of infections caused by various microorganisms, in particular drug resistant microorganisms.

### BACKGROUND OF THE INVENTION

For years, the discovery and use of antimicrobial agents has remained the most successful strategy in the fight against infectious diseases caused by microorganisms. However, such a dependence on antimicrobial agents and their overuse has resulted in disastrous consequences: the emergence and spread of microorganisms that are resistant to cheap and effective “first-line” antimicrobial agents. Nowadays, a significant fraction of microorganisms that cause infections are resistant to at least one of the antimicrobial agents most commonly used for the treatment.

The World Health Organization Fact Sheet (No. 194, revised January 2002) notes that the bacterial infections which contribute most to human diseases are also those in which emerging and microbial resistance is most evident: diarrhoeal diseases, respiratory tract infections, meningitis, sexually transmitted infections, and hospital-acquired infections. Some important examples of microorganisms resistant to antimicrobial agents include: penicillin-resistant Streptococcus pneumoniae, vancomycin-resistant enterococci, methicillin-resistant Staphylococcus aureus, multi-resistant salmonellae, Klebsiella, Escherichia coli, Enterobacter, Serratia, P. aeruginosa, and multi-resistant Mycobacterium tuberculosis.

The problem of emerging drug-resistance in microorganisms is often tackled by switching to next-line of antimicrobial agents, which can be more expensive and sometimes more toxic. However, even this may not be a permanent solution and the microorganisms often develop resistance to the newer antimicrobial agents in due course. Bacteria are particularly efficient in developing resistance, because of their ability to multiply very rapidly and pass on the resistance genes as they replicate.

Several antimicrobial combinations have been studied in the prior art including those by Mayer et al. (Investigation of the aminoglycosides, fluoroquinolones and third-generation cephalosporin combinations against clinical isolates of Pseudomonas spp. J. Antimicrob. Chemother., 43, 651-657, 1999); Gradelski et al. (Synergistic activities of gatifloxacin in combination with other antimicrobial agents against clinical isolates of Pseudomonas aeruginosa and related species. Antimicrob. Agents Chemother., 45, 3220-3222, 2001); Fish et al. (Synergistic activity of cephalosporins plus fluroquinolones against Pseudomonas aeruginosa with resistance to one or both drugs. J. Antimicrob. Chemother., 50, 1045-1049, 2002) and Davis et al. (In vitro activity of gatifloxacin alone and in combination with cefepime, meropenem, piperacillin and gentamicin against multidrug-resistant organisms, J. Antimicrob. Chemother., 51, 1203-1211, 2003). Fish et al. found combination of cefepime or ceftazidime with ciprofloxacin, levofloxacin, gatifloxacin or moxifloxacin synergistic against 10 clinical Pseudomonas aeruginosa strains including those resistant to both cephalosporins and flouroqinolones. In another study, N. Sivagurunathan et al. (Synergy of gatifloxacin with cefoperazone and cefoperazone-sulbactam against resistant strains of Pseudomonas aeruginosa. J. Medical Microb., 57, 1514-1517, 2008) obtained in vitro synergy with gatifloxacin and cefoperazone and gatifloxacin-cefoperazone-sulbactam combination against resistant strains of Pseudomonas aeruginosa. In few cases, these antibiotic combinations have also been successfully employed as an effective treatment for Pseudomonas aeruginosa nosocomial infections (Al-Hasan et.al, β-Lactam and Fluroquinolonecombination antibiotic therapy for bateremia caused by Gram-negative bacilli. Antimicrob. Agents Chemother., 53(4), 1386-1394, 2009). Bacalum et al. have shown that ceftazidime binds to outer membrane porins with high affinity (Bacalum et al. Romanian. J. Biophys., 19, 105-116, 2009).

Microorganisms use several mechanisms to acquire resistance to antimicrobial agents including, such as for example, drug inactivation or modification (e.g. enzymatic deactivation of Penicillin G in some penicillin-resistant bacteria through the production of β-lactamases), alteration of target site (e.g. alteration of PBP, the binding target site of penicillins in MRSA and other penicillin-resistant bacteria), alteration of metabolic pathway (e.g. some sulfonamide-resistant bacteria do not require para-aminobenzoic acid (PABA), an important precursor for the synthesis of folic acid and nucleic acids in bacteria inhibited by sulfonamides) or reduced accumulation of antimicrobial agents through efflux pumps (e.g. by decreasing permeability and/or increasing active efflux of the antimicrobial agents across the cell surface).

Efflux pumps transport substrate molecules, including antimicrobial agents, from cytoplasm in an energy-dependent manner. Such a removal of antimicrobial agent from the microorganism results in lowering of the effective concentrations of the antimicrobial agent within the microorganism and consequently results in substantial reduction in antimicrobial activity of such agent. Efflux pump inhibitors inhibit various cellular efflux pumps of microorganism and are useful, for example, in treating microbial infections by reducing transport of antimicrobial agent or by preventing the transport of a compound synthesized by microorganism (useful in improving their growth and/or maintenance). Efflux is also believed to act as a predisposing step for additional acquisition of resistance through target modification involving mutations. It is noteworthy that frequency of efflux-mediated resistance is higher as compared to target site mutations. Multi-drug efflux pumps are expressed in both gram-positive as well as gram-negative bacteria, but it is in gram-negatives that they exert their therapeutically disastrous consequences through change in the drug susceptibilities by several folds. For example, prevalence of efflux pump overproduction in clinical strains of Pseudomonas aeruginosa, an important pathogen, which is highly resistant to a variety of antibiotic therapy, may range from 14-75%. Fluoroquinolones, β-lactams and aminoglycosides are primary agents available for the treatment of infections caused by this pathogen. Several multi drug resistance conferring efflux pumps such as Acr AB-TolC RND (resistance nodulation division) pumps have been characterized in E. coli and Pseudomonas. Additionally four multi-component MexA-MexB-OprM, MexC-MexD-OprJ, MexE-MexF-OprN, and MexX-MexY-OprM MDR RND pumps are reported in P. aeruginosa and other organisms. The proton motive force drives substrate extrusion or export by these pumps and recent data indicates that substrates may be pumped from the periplasm or the inner leaflet of the cytoplasmic membrane. These pumps have overlapping spectra of antibiotic substrates. For example, all four pumps confer varying degree of resistance to fluoroquinolones and many other antibacterial agents and mutants that over express these pumps have been isolated in clinical settings.

Yet another way the microorganisms can acquire resistance to antimicrobial resistance is through modification of various proteins in the outer membrane, which control the entry of foreign substances (including antimicrobial agents) into the microorganism body, for example, by decreasing permeability. This mechanism is in particular of interest in microorganisms wherein the outer membrane provides barrier for the entry of antimicrobial agents, for example gram-negative bacteria.

Porins are a type of Outer Membrane Proteins (OMP) present in the outer membrane of gram-negative bacteria that are capable of forming channels and allow diffusion of hydrophilic solutes across the outer membrane. The loss of ability of porins to transport the antimicrobial agents into the microorganism is one of the various mechanism by which the microorganisms can acquire resistance to antimicrobial agents. For example, the loss or deficiency of required porins can reduce the outer membrane permeability of antimicrobial agents. In Gram-negative bacteria, the outer membrane limits the rate of antimicrobial agents entering the cell and the efflux pumps actively export antimicrobial agents out of the bacteria. Efflux transporters are expressed in all living cells, protecting them from the toxic effects of organic chemicals. The antimicrobial agents expelled out of the cell have to cross the low permeability outer membrane in order to enter again; therefore the efflux pumps work synergistically with the low permeability of the outer membrane. An increased efflux of antibiotic from the bacterium produces a reduction in drug accumulation and an increment in the MIC.

Porin modulators can enhance activity of porins advantageously and facilitate entry of antimicrobial agents into the microorganism body (for example, bacterial cell), which provides higher concentration of the antimicrobial agent in the microorganisms increasing its efficacy.

Development of novel agents to overcome multi-drug efflux systems has so far met with limited success. For example, the glycylcycline antibiotic, tigecycline originally thought to be unaffected by tetracycline-specific Tet efflux, is a substrate for MexA-MexB-OprM, MexC-MexD-OprJ, and MexX-MexY-OprM efflux pumps in Pseudomonas severely compromising its effectiveness against this pathogen. Moreover, borderline activity of tigecycline against genus Proteus is due to AcrAB multidrug efflux system operating in this organism. In fact, gram-negative efflux transporters effectively extrude all the novel antibacterials studied so far. It is therefore, that inhibition of efflux pump continues to be an attractive strategy in significantly improving the clinical performance of antimicrobial agents by decreasing the intrinsic resistance and reversing the acquired resistance.

The present inventors have surprisingly found that β-lactam compounds can act as efficient efflux pump inhibitors and/or proin modulators and restore activity of various antimicrobial agents in a wide variety of microorganisms. The use of β-lactam compounds as efflux-pump inhibitors and/or porin modulators has been unexpectedly found to control and/or reverse drug resistance in microorganisms, even in highly resistant microorganisms.

### SUMMARY OF THE INVENTION

The invention relates to efflux pump inhibitors and their use in treating infections caused by microorganisms or reducing resistance of microorganisms to antimicrobial agents. The invention also relates to pharmaceutical compositions and their use in treating infections caused by microorganisms.

In one general aspect, there is provided a method of inhibiting efflux pump activity in a microorganism, comprising contacting said microorganism with an effective amount of an efflux pump inhibitor, wherein said efflux pump inhibitor is a β-lactam compound.

In another general aspect, there is provided a method of modulating porin activity in a microorganism, comprising contacting said microorganism with an effective amount of a porin modulator, wherein said porin modulator is a β-lactam compound.

In another general aspect, there is provided a method of treating infection caused by a microorganism in a subject, comprising administering to the subject in need thereof, a therapeutically effective amount of an efflux pump inhibitor in combination with at least one antimicrobial agent, wherein said efflux pump inhibitor is a β-lactam compound.

In another general aspect, there is provided a method for prophylactic treatment of a subject, comprising administering to a subject at risk of infection caused by microorganism, a prophylactically effective amount of an efflux pump inhibitor, wherein said efflux pump inhibitor is a β-lactam compound.

In another general aspect, there is provided a method for prophylactic treatment of a subject, comprising administering to a subject at risk of infection by microorganism, a prophylactically effective amount of an efflux pump inhibitor in combination with at least one antimicrobial agent, wherein said efflux pump inhibitor is a β-lactam compound.

In another general aspect, there is provided a pharmaceutical composition effective for treatment of infection in a subject caused by a microorganism, comprising an efflux pump inhibitor in combination with at least one antimicrobial agent, wherein said efflux pump inhibitor is a β-lactam compound.

The details of one or more embodiments of the inventions are set forth in the description below. Other features, objects and advantages of the inventions will be apparent from the following description including claims.

### DETAILED DESCRIPTION OF THE INVENTION

Reference will now be made to the exemplary embodiments, and specific language will be used herein to describe the same. It should nevertheless be understood that no limitation of the scope of the invention is thereby intended. Alterations and further modifications of the inventive features illustrated herein, and additional applications of the principles of the inventions as illustrated herein, which would occur to one skilled in the relevant art and having possession of this disclosure, are to be considered within the scope of the invention. It must be noted that, as used in this specification and the appended claims, the singular forms “a,” “an,” and “the” include plural referents unless the content clearly dictates otherwise.

The inventors have discovered that β-lactam compounds are capable of increasing intracellular concentration of antimicrobial agents by inhibiting or by dysfunction of cellular efflux pumps in microorganisms or by modulating porin activity in a microorganism. Such efflux pumps export substrate molecules, including antimicrobial agents, from the cytoplasm in an energy-dependent or independent manner thereby displaying resistance to antimicrobial agents. Such efflux pump inhibitors are useful, for example, in treating infections caused by microorganisms by reducing export of co-administered antimicrobial agents. Also disclosed herein are compositions that include β-lactam compounds as efflux pump inhibitors and methods of treating infections caused by microorganisms using such compositions. The β-lactam compounds according to the present invention can also act as porin modulators, and can enhance porin activity in a microorganism, which results in increase in intracellular concentration of antimicrobial agents in the microorganism.

The term “inhibition”, “inhibits”, “inhibiting” and “inhibitor” as used herein refer to a compound that prohibits or a method of prohibiting or dysfunctioning of a specific action or function. For example, the term “inhibiting a microorganism”, as used herein refers to reducing or preventing growth of the microorganism, or preventing the microorganism from attaching to normal cells, and/or the elimination of some or all of the infectious particles or infecting microbial cells from the subject being treated. The term “inhibiting efflux pump activity” as used herein refers to prevention, suppression, dysfunction or reduction of the efflux-pump activity. It may not be necessary that “inhibition or inhibiting efflux pump activity” should mean completely blocking of the efflux pump activity, but also means reducing the efflux pump activity by a sufficient degree to enable the desired effect to be achieved. The term “efflux pump inhibition” or “efflux pump inhibitor” also includes “porin modulation” or “porin modulator”. The porin modulators enhance the ability of porins to effectively transport the antimicrobial agents into the microorganism, which otherwise is not possible or is reduced due to the resistance acquired by the microorganism to the antimicrobial agent. The efflux pump inhibitors according to the present invention can advantageously act as porin modulators.

The term “efflux pump” as used herein refers to a protein assembly, which transports or exports substrate molecules from the cytoplasm or periplasm of a cell, in an energy-dependent or independent fashion. The term “efflux pump activity” as used herein refers to a mechanism responsible for export of substrate molecules, including antimicrobial agents, outside the cell. The term “efflux pump inhibitor” as used herein refers to a compound, which interferes with the ability of an efflux pump to transport or export a substrate, including antimicrobial agent.

The term “microorganism” or “microbe” as used herein includes bacteria, fungi, protozoa, yeast, mold, and mildew.

The term “contacting” as used herein refers to positioning, applying or addition of efflux pump inhibitor according to the present invention in such a way that it is in direct or indirect contact with the microorganism or its cell(s), completely or partially. It will be appreciated by those skilled in the art that such “contacting” can be achieved in many ways, including for example, surface application, bulk seeding or addition of the efflux pump inhibitor in the test system, bulk seeding at the desired surface or administration into the body of the subject, where the microorganism is likely to be present, in such a way that the efflux pump inhibitor is likely to come into direct or indirect contact with microorganism, either completely or partially.

The term “infection” as used herein includes presence of a microorganism in or on a subject, which, if its growth were inhibited, would result in a benefit to the subject. As such, the term “infection” in addition to referring to the presence of microorganisms also refers to normal flora, which are not desirable. The term “infection” includes infection caused by bacteria, fungi, protozoa, yeast, mold, or mildew.

The term “treat”, “treating” or “treatment” as used herein refers to administering a pharmaceutical composition for prophylactic and/or therapeutic purposes. The term “prophylactic treatment” refers to treating a subject who is not yet infected, but who is susceptible to, or otherwise at a risk of infection. The term “therapeutic treatment” refers to administering treatment to a subject already suffering from infection. Thus, in preferred embodiments, treating is the administration to a subject (either for therapeutic or prophylactic purposes) of therapeutically effective amount of efflux pump inhibitor alone, or in combination with one or more antimicrobial agents, either simultaneously or serially.

The term “administration” or “administering” includes delivery to a subject, including for example, by any appropriate method, which serves to deliver the drug to the site of the infection. The method of administration can vary depending on various factors, such as for example, the components of the pharmaceutical composition, the site of the potential or actual infection, the microorganism involved, severity of the infection, age and physical condition of the subject. Some non-limiting examples of ways to administer a composition or a compound to a subject according to this invention include oral, intravenous, topical, intrarespiratory, intraperitoneal, intramuscular, parenteral, sublingual, transdermal, intranasal, aerosol, intraocular, intratracheal, intrarectal, vaginal, gene gun, dermal patch, eye drop, ear drop or mouthwash

The term “subject” as used herein refers to vertebrate or invertebrate, including a mammal The term “subject” includes human, animal, a bird, a fish, or an amphibian.

The term “therapeutically effective amount” as used herein refers to an amount, which has a therapeutic effect or is the amount required to produce a therapeutic effect in a subject. For example, a therapeutically effective amount of an efflux pump inhibitor and/or antimicrobial agent is the amount of the efflux pump inhibitor and/or antimicrobial agent required to produce a desired therapeutic effect as may be judged by clinical trial results, model animal infection studies, and/or in vitro studies (e.g. in agar or broth media). The therapeutic amount depends on several factors, including but not limited to, the microorganism involved, characteristics of the subject (for example height, weight, sex, age and medical history), severity of infection and the particular efflux pump inhibitor and/or antimicrobial agent used. For prophylactic treatments, a therapeutically or prophylactically effective amount is that amount which would be effective to prevent a microbial infection.

The term “growth” as used herein refers to the growth of microorganisms and includes reproduction or population expansion of the microorganism. The term also includes maintenance of on-going metabolic processes of a microorganism, including processes that keep the microorganism alive.

The term “synergistic” or “synergy” as used herein refers to the interaction of two or more agents so that their combined effect is greater than their individual effects.

The term “antimicrobial agent” as used herein refers to compounds capable of inhibiting, reducing or preventing growth of a microorganism, capable of inhibiting or reducing ability of a microorganism to produce infection in a host, or capable of inhibiting or reducing ability of a microorganism to multiply or remain infective in the environment. The term “antimicrobial agent” also refers to compounds capable of decreasing infectivity or virulence of a microorganism. Antimicrobial agents according to this invention include antibiotic agents, antibacterial agents and antifungal agents.

The term “antibacterial agent” as used herein refers to compounds capable of inhibiting, reducing or preventing growth of bacteria, capable of inhibiting or reducing ability of bacteria to produce infection in a host, or capable of inhibiting or reducing ability of bacteria to multiply or remain infective in the environment. The term “antibacterial agent” also refers to compounds capable of decreasing infectivity or virulence of bacteria.

The term “antifungal agent” as used herein refers to compounds capable of inhibiting, reducing or preventing growth of fungi, capable of inhibiting or reducing ability of fungi to produce infection in a host, or capable of inhibiting or reducing ability of fungi to grow or remain infective in the environment. The term “antifungal agent” also refers to compounds capable of decreasing infectivity of fungi.

The term “compound” as used herein refers to and includes various pharmaceutically acceptable forms of the active ingredient including, without any limitation, pharmaceutically acceptable salts, pro-drugs, metabolites, esters, ethers, hydrates, polymorphs, solvates, complexes, enantiomers, adducts etc. For example, the term “cephalosporin compound” includes various pharmaceutically acceptable forms of cephalosporin active ingredient including, without any limitation, pharmaceutically acceptable salts, pro-drugs, metabolites, esters, ethers, hydrates, polymorphs, solvates, complexes, enantiomers, adducts etc.

The term “β-lactam” compound as used herein refers to a class of natural or synthetic compounds having β-lactam nucleus. Non-limiting examples of the β-lactam compounds according to this invention include cephalosporins, cephamycins, penicillins, and carbapenem compounds.

A “carrier” or “excipient” is a compound or material used to facilitate administration of a compound, for example, to increase the solubility of the compound. Solid carriers include, e.g., starch, lactose, dicalcium phosphate, sucrose, and kaolin. Liquid carriers include, e.g., sterile water, saline, buffers, non-ionic surfactants, and edible oils such as oil, peanut and sesame oils. In addition, various adjuvants such as are commonly used in the art may be included. These and other such compounds are described in the literature, e.g., in the Merck Index, Merck & Company, Rahway, N.J. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press.

In one embodiment, there are provided β-lactam compounds as efflux pump inhibitors and/or porin modulators.

In some embodiments, there is provided a method of inhibiting efflux pump activity in a microorganism, comprising contacting said microorganism with an effective amount of an efflux pump inhibitor, wherein said efflux pump inhibitor is a β-lactam compound, generically or specifically described herein.

In some other embodiments, there is provided a method of modulating porin activity in a microorganism, comprising contacting said microorganism with an effective amount of a porin modulator, wherein said porin modulator is a β-lactam compound.

In some other embodiments, there is provided a method of treating infection caused by microorganisms in a subject, comprising administering to the subject in need thereof, a therapeutically effective amount of an efflux pump inhibitor in combination with at least one antimicrobial agents, wherein said efflux pump inhibitor is a β-lactam compound, described generically or specifically herein.

In some embodiments, a method is provided a method for treating infection caused by microorganisms in a subject, including humans and animals, by treating a subject suffering from such infection with at least one antimicrobial agents in combination with an efflux pump inhibitor, which increases the susceptibility of the microorganism for that antimicrobial agent, such efflux pump inhibitors being a β-lactam compound, generically or specifically described herein. In this way a microorganism causing the infection can be treated using the antimicrobial agent in smaller quantities, or can be treated with an antimicrobial agent, which is therapeutically ineffective when used in the absence of the efflux pump inhibitor. Thus, this method of treatment is especially useful for the treatment of infections involving microorganisms that are difficult to treat using an antimicrobial agent alone due to a need for high dosage levels (which can cause undesirable side effects), or due to lack of any clinically effective antimicrobial agents or antimicrobial activity. Alternatively, such a method may also be advantageously used for treating infections involving microorganisms that are susceptible to particular antimicrobial agents as a means to reduce the dosage of those particular agents and/or increase the effectiveness of the agents. This can reduce the risk of side effects. The method is also useful for treating infections involving microorganisms that are susceptible to particular antimicrobial agents as a way of reducing the frequency of selection of resistant microbes.

In some embodiments, the use of β-lactam compounds, described generically or specifically herein, as inhibitors of efflux pump activity is in particular very useful in treating infections caused by microorganisms, that have developed resistance to one or more antimicrobial agents due to efflux pump activity. In such cases, the use of β-lactam compounds as inhibitors of efflux pump activity lower or eliminate the resistance of such resistant microorganism and makes them susceptible for treatment with antimicrobial agents, including those previously not effective or less effective.

In some embodiments, there is provided a method for prophylactic treatment of a subject. This method comprises administering to a subject at risk of infection caused by microorganisms, a prophylactically effective amount of an efflux-pump inhibitor, alone or in combination with at least one antimicrobial agents, wherein said efflux-pump inhibitor is a β-lactam compound, generally or specifically described herein.

In some embodiments, a method is provided for enhancing the antimicrobial activity of antimicrobial agents against microorganisms, in which such microorganism is contacted with an efflux pump inhibitor, and optionally one or more antimicrobial agents, wherein the efflux pump inhibitor is a β-lactam compound, described generally or specifically herein. This method makes an antimicrobial agent more effective against microorganism, which expresses efflux pump or exhibits efflux pump activity. Such methods are particularly effective in treating infections caused by microorganism that express efflux pump or exhibit efflux pump activity as a means to develop resistance against the action of the antimicrobial agent.

In some other embodiments, a method is provided for suppressing growth of microorganisms capable of expressing a multi-drug resistance efflux pump. The method generally involves contacting such microorganism with an efflux pump inhibitor, in the presence of one or more antimicrobial agents, wherein the efflux pump inhibitor is a β-lactam compound, described generally or specifically herein.

In other embodiments, any of the β-lactam compounds generically or specifically described herein may be administered as an efflux pump inhibitor either alone or, in combination with one or more therapeutic agents, including antimicrobial agents.

In some other embodiments, there are provided pharmaceutical compositions effective for treatment of infection in a subject caused by a microorganism, comprising an efflux pump inhibitor in combination with at least one antimicrobial agent, wherein said efflux pump inhibitor is a β-lactam compound.

In some embodiments, a subject is identified as infected or is identified as at a risk of infection by microorganism, that are resistant to or are capable of developing resistance to one or more antimicrobial agents. The subject may then be treated with the antimicrobial agent in combination with a β-lactam compound, generally or specifically described herein, and acting as an inhibitor of efflux pump activity compound disclosed herein.

In some embodiments the efflux pump inhibitor used in methods or composition described herein is a β-lactam compound, described generally or specifically herein. In some other embodiments, the efflux pump inhibitor used in methods or compositions described herein, is ceftazidime or cefepime.

The amount of efflux pump inhibitor and/or antimicrobial agent, when administered as a pharmaceutical composition or otherwise, according to this invention is sufficient to provide the desired therapeutic effect, including for example: elimination, control, suppression or reduction of infection caused by microorganism; elimination, control, suppression or reduction in occurrence or presence of efflux mechanism resulting in resistance in microorganism to one or more antimicrobial agents; prophylactic treatment of a subject at a risk of infection caused by one or more microorganisms. The therapeutic amount depends on several factors, including but not limited to, in vitro and/or in vivo test system involved, the particular microorganism involved, characteristics of the subject (for example height, weight, sex, age and medical history), severity of infection and the particular efflux pump inhibitor and/or antimicrobial agent used. For prophylactic treatments, a therapeutically or prophylactically effective amount is that amount which would be effective to prevent a microbial infection. In general, the amount of inhibitor of efflux activity and/or antimicrobial agent and mode of administration largely depend on the extent and duration of the therapeutic response desired in terms of inhibition of the efflux pump activity and/or treatment of infection and can vary depending on various factors, including nature of the microorganism and its population. If desired, one or more of other pharmaceutically acceptable substances may also be used in combination with the inhibitors of efflux pump activity and/or one or more antimicrobial agents.

The amount of β-lactam compound that needs to be administered as an inhibitor of efflux activity and its mode of administration largely depend on the extent and duration of the therapeutic response desired in terms of inhibition of the efflux pump activity and can vary depending on various factors, including nature of the test system, microorganism and its population. If desired, one or more of other pharmaceutically acceptable substances may also be used in combination with the inhibitors of efflux pump activity. In some embodiments, an efflux pump inhibitor is administered at a level sufficient to overcome or suppress the emergence of efflux pump-mediated resistance in bacteria. In some embodiments, this level produces the effective efflux pump inhibitory concentration at the site of infection. In other embodiments, this level produces an effect equivalent to shutting down all efflux pumps in the microorganism.

In general, in methods according to this invention, the efflux pump inhibitor, alone or in combination one or more antimicrobial agents, either in the form of a pharmaceutical composition or otherwise, is administered by any appropriate method, which serves to deliver the efflux pump inhibitor and/or antimicrobial agent to the site of the infection. The method of administration can vary depending on various factors, such as for example, the components of the pharmaceutical composition, the site of the potential or actual bacterial infection, the microorganism involved, severity infection, age and physical condition of the subject. The β-lactam compound and/or one or more antimicrobial agents may be administered either simultaneously or sequentially and by the same or different route of administration. Some non-limiting examples of administering the composition to a subject according to this invention include oral, intravenous, topical, intrarespiratory, intraperitoneal, intramuscular, parenteral, sublingual, transdermal, intranasal, aerosol, intraocular, intratracheal, intrarectal, vaginal, gene gun, dermal patch, eye drop, ear drop or mouthwash.

The β-lactam compounds according to this invention include natural or synthetic compounds having β-lactam nucleus. Typical, Non-limiting examples of the β-lactam compounds according to this invention include cephalosporins, cephamycins, penicillins, and carbapenem compounds.

Typical, non-limiting examples of cephalosporins and cephamycins include cefazolin, cefacetrile, cefadroxil, cefalexin, cefaloglycin, cefalonium, cefaloridine, cefalotin, cefapirin, cefatrizine, cefazedone, cefazaflur, cefradine, cefroxadine, ceftezole, cefaclor, cefamandole, cefminox, cefonicid, ceforanide, cefotiam, cefprozil, cefbuperazone, cefuroxime, cefuzonam, cephamycin, cefoxitin, cefotetan, cefmetazole, carbacephem, cefixime, ceftazidime, ceftriaxone, cefcapene, cefdaloxime, cefdinir, cefditoren, cefetamet, cefmenoxime, cefodizime, cefoperazone, cefotaxime, cefpimizole, cefpiramide, cefpodoxime, cefsulodin, cefteram, ceftibuten, ceftiolene, ceftizoxime, oxacephem, cefepime, cefozopran, cefpirome, cefquinome, ceftobiprole, ceftiofur, cefquinome, cefovecin, ceftaroline, ceftobiprole, CXA-101 (CAS Registry No. 936111-69-2, CA Index Name: 1H-Pyrazolium, 5-amino-4-[[[(2-aminoethyl)amino]carbonyl]amino]-2-[[(6R,7R)-7-[[(2Z)-2-(5-amino-1,2,4-thiadiazol-3-yl)-2-[(1-carboxy-1-methylethoxy)imino]acetyl]amino]-2-carboxy-8-oxo-5-thia-1-azabicyclo-[4.2.0] oct-2-en-3-yl]methyl]-1-methyl-) etc.

Typical, non-limiting examples of penicillins include amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, mecillinam etc.

Typical, non-limiting examples of carbapenem compounds include ertapenem, doripenem, imipenem, meropenem, sulopenem etc.

Other non-limiting examples of β-lactam compounds according to this invention include monocyclic β-lactam compounds such as aztreonam, nocardicin etc.

In some embodiments, the β-lactam compound is ceftazidime or cefepime.

According to this invention, the microorganisms include one or more of bacteria, fungi, protozoa, yeast, mold, and mildew.

Typical, non-limiting examples of bacteria include Pseudomonas aeruginosa, Pseudomonas fluorescens, Pseudomonas acidovorans, Pseudomonas alcaligenes, Pseudomonas putida, Stenotrophomonas maltophilia, Burkholderia cepacia, Aeromonas hydrophilia, Escherichia coli, Citrobacter freundii, Salmonella typhimurium, Salmonella typhi, Salmonella paratyphi, Salmonella enteritidis, Shigella dysenteriae, Shigella flexneri, Shigella sonnei, Enterobacter cloacae, Enterobacter aerogenes, Klebsiella pneumoniae, Klebsiella oxytoca, Serratia marcescens, Francisella tularensis, Morganella morganii, Proteus mirabilis, Proteus vulgaris, Providencia alcalifaciens, Providencia rettgeri, Providencia stuartii, Acinetobacter calcoaceticus, Acinetobacter haemolyticus, Yersinia enterocolitica, Yersinia pestis, Yersinia pseudotuberculosis, Yersinia intermedia, Bordetella pertussis, Bordetella parapertussis, Bordetella bronchiseptica, Haemophilus influenzae, Haemophilus parainfluenzae, Haemophilus haemolyticus, Haemophilus parahaemolyticus, Haemophilus ducreyi, Pasteurella multocida, Pasteurella haemolytica, Branhamella catarrhalis, Helicobacter pylori, Campylobacter fetus, Campylobacter jejuni, Campylobacter coli, Borrelia burgdorferi, Vibrio cholerae, vibrio parahaemolyticus, Legionella pneumophila, Listeria monocytogenes, Neisseria gonorrhoeae, Neisseria meningitidis, Gardnerella vaginalis, Bacteroides fragilis, Bacteroides distasonis, Bacteroides 3452A homology group, Bacteroides vulgatus, Bacteroides ovalus, Bacteroides thetaiotaomicron, Bacteroides uniformis, Bacteroides eggerthii, Bacteroides splanchnicus, Clostridium difficile, Mycobacterium tuberculosis, Mycobacterium avium, Mycobacterium intracellulare, Mycobacterium leprae, Corynebacterium diphtheriae, Corynebacterium ulcerans, Streptococcus pneumoniae, Streptococcus agalactiae, Streptococcus pyogenes, Enterococcus faecalis, Enterococcus faecium, Staphylococcus aureus, Staphylococcus epidermidis, Staphylococcus saprophyticus, Staphylococcus intermedius, Staphylococcus hyicus subsp. hyicus, Staphylococcus haemolyticus, Staphylococcus hominis and Staphylococcus saccharolyticus.

Typical, non-limiting examples of fungi include those causing candidiasis, thrush, cryptococcosis, histoplasmosis, blastomycosis, aspergillosis, coccidioidomycosis, paracoccidiomycosis, sporotrichosis, zygomycosis, chromoblastomycosis, lobomycosis, mycetoma, onychomycosis, piedra pityriasis versicolor, tinea barbae, tinea capitis, tinea corporis, tinea cruris, tinea favosa, tinea nigra, tinea pedis, otomycosis, phaeohyphomycosis, or rhinosporidiosis.

Antimicrobial agents according to this invention include antibiotic agents, antibacterial agents and antifungal agents.

Typical, non-limiting examples of antibacterial agents include aminoglycoside, oxazolidinone, quinolone, ansamycin, carbacephem, carbapenem, cephalosporin, glycopeptide, macrolide, penicillin, polypeptide antibacterial agents etc.

Typical, non-limiting examples of aminoglycoside antibacterial agents according to this invention include amikacin, gentamicin, kanamycin, neomycin, netilmicin, streptomycin, tobramycin, paromomycin etc.

Typical, non-limiting examples of oxazolidinone antibacterial agents according to this invention include linezolid, ranbezolid, torezolid, radezolid etc.

Typical, non-limiting examples of quinolone antibacterial agents according to this invention include cinoxacin, flumequine, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balofloxacin, gatifloxacin, grepafloxacin, levofloxacin, moxifloxacin, pazufloxacin, sparfloxacin, temafloxacin, tosufloxacin, clinafloxacin, gemifloxacin, sitafloxacin, trovafloxacin, prulifloxacin, garenoxacin, delafloxacin, danofloxacin, difloxacin, enrofloxacin, ibafloxacin, marbofloxacin, orbifloxacin, sarafloxacin, nemonoxacin, finafloxacin, delafloxacin etc.

Typical, non-limiting examples of ansamycin antibacterial agents according to this invention include geldanamycin, herbimycin etc.

Typical, non-limiting examples of carbacephem antibacterial agents according to this invention include loracarbef etc.

Typical, non-limiting examples of carbapenem antibacterial agents according to this invention include ertapenem, doripenem, imipenem, meropenem, sulopenem etc.

Typical, non-limiting examples of cephalosporin antibacterial agents according to this invention include cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, ceftarolin, CXA-101 (CAS Registry No. 936111-69-2, CA Index Name: 1H-Pyrazolium, 5-amino-4-[[[(2-aminoethyl)amino]carbonyl]amino]-2-[[(6R,7R)-7-[[(2Z)-2-(5-amino-1,2,4-thiadiazol-3-yl)-2-[(1-carboxy-1-methylethoxy)imino]acetyl]amino]-2-carboxy-8-oxo-5-thia-l-azabicyclo-[4.2.0]oct-2-en-3-yl]methyl]-1-methyl-) etc.

Typical, non-limiting examples of glycopeptide antibacterial agents according to this invention include teicoplanin, vancomycin, dalbavancin, telavancin, oritavancin etc.

Typical, non-limiting examples of macrolide antibacterial agents according to this invention include azithromycin, clarithromycin, dirithromycin, erythromycin, roxithromycin, troleandomycin, telithromycin, spectinomycin, CEM 101 (CAS Registry No. 1159405-40-9), Modithromycin (CAS 736992-12-4, also known as EDP 420).

Typical, non-limiting examples of penicillin antibacterial agents according to this invention include amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin, mecillinam etc.

Typical, non-limiting examples of polypeptide antibacterial agents according to this invention include bacitracin, colistin, polymyxin-B etc.

Typical, non-limiting examples of sulfonamide antibacterial agents according to this invention include mafenide, sulfonamidochrysoidine, sulfacetamide, sulfadiazine, sulfamethizole, sulfanilimide, sulfasalazine, sulfisoxazole, trimethoprim, trimethoprim-sulfamethoxazole etc.

Typical, non-limiting examples of tetracycline antibacterial agents according to this invention include demeclocycline, doxycycline, minocycline, oxytetracycline, tetracycline, tigecycline, amadacycline (CAS Registry No. 389139-89-3, also known as PTK-0796) etc.

Other examples of typical antibacterial agents according to this invention include arsphenamine, chloramphenico, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin, dalfopristin, rifampicin, thiamphenicol, tinidazole, dapsone, clofazimine, aztreonam, nocardicin, clavulanic acid, tazobactam, sulbactam, NXL104 (CAS Registry No. 1192491-61-4) etc.

Typical, non-limiting examples of antifungal agents according to this invention include polyene, imidazole, triazole, thiazole, allylamine, and echinocandin compounds.

Typical, non-limiting examples of antifungal agents according to this invention include polyene antifungal agents (such as natamycin, rimocidin, filipin, nystatin, amphotericin b, candicin, hamycin etc.); imidazoles (such as miconazole, ketoconazole, clotrimazole, econazole, bifonazole, butoconazole, fenticonazole, isoconazole, oxiconazole, sertaconazole, sulconazole, tioconazole, griseofulvin etc.); triazoles (such as fluconazole, itraconazole, isavuconazole, ravuconazole, posaconazole, voriconazole, terconazole etc.); thiazoles (such as abafungin); allylamines (such as terbinafine, amorolfine, naftifine, butenafine etc); echinocandins (such as anidulafungin, caspofungin, micafungin etc) and other antifungal agents including benzoic acid, ciclopirox, tolnaftate, undecylenic acid, 5-fluorocytosine, haloprogin, sodium bicarbonate, allicin, tea tree oil, citronella oil, iodine, olive leaf, orange oil, palmarosa oil, patchouli, lemon myrtle, neem seed oil, coconut oil, zinc, selenium etc.

In some embodiments, the antimicrobial agent is a fluoroquinolone of general Formula I:

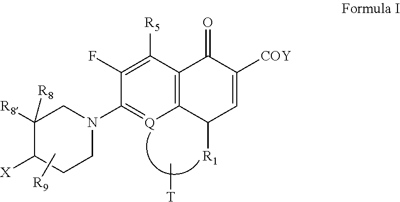

### Wherein;

R₁ is C₁₋₅ alkyl being unsubstituted or substituted with from 1 to 3 fluoro atoms, C₃-₆ cycloalkyl being unsubstituted or substituted with from 1 to 2 fluoro atoms, or aryl being unsubstituted or substituted with from 1 to 3 fluoro atoms;
or when Q is CH and the nitrogen atom to which R₁ is linked forms an optionally substituted 5-, 6- or 7-membered ring with the carbon atom of Q, the ring optionally containing one or more hetero atoms selected from nitrogen, oxygen or sulfur atoms, said heteroatom(s) represented by T, preferably R₁ is CH₂CH₂—, CH₂T—, CH₂CH₂CH₂—, CH₂CH₂T—, CH₂TCH₂—, TCH₂T—, TCH₂CH₂CH₂CH₂— CH₂CH₂CH₂T—, CH₂TCH₂CH₂—, or TCH₂CH₂T— where T represents NH, O, or S. This 5- to 7-membered ring may be substituted with 1 or 2 of the same substituents as those defined above for R₁, preferably by one C₁-C₅ alkyl group.
Y is OR₃ where
R₃ is hydrogen;
R₃ is C₁-C₂₀ alkyl, such as straight chain or branched chain aliphatic residues such as methyl, ethyl, propyl, isopropyl, butyl, isobutyl, tertiary butyl, pentyl, hexyl or their branched chain isomers;
R₃ is aralkyl such as benzyl, phenethyl, or phenylpropyl;

### R₃ is CH₂CH(NH₂)COOH;

R₃ is (CH₂)ₙCHR₁₀—OCOR₁₁ or (CH₂)ₙ—CHR₁₀—OCO₂R₁₁ wherein R₁₀ is H, or CH₃; n is 0-3 and R₁₁ is C₁-C₂₀ alkyl as hereinbefore defined, or substituted C₁-C₆ alkyl with substituents such as hydroxy, halogen, amino, or mercapto; or aralkyl such as benzyl, phenethyl, phenylpropyl or R₁₁ is

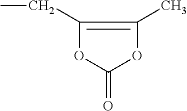

or R₃ is ∀-aminoalkanoyl such as ∀-aminopropionyl or R₃ is alkanoylalkyl group such as acetoxymethyl, acetoxyethyl, pivaloyloxy-methyl, or pivaloyloxyethyl group; or R₃ is

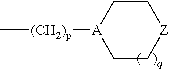

wherein;
A is CH or N, and when A is CH, Z is NH or NCH₃, and when A is N, Z is CH, O, NH, S, or NCH₃; p is 0-2; q is 0-2, preferably it is a group such as N-methylpiperidin-4-yl, pyrrolidin-2-yl-ethyl, piperidin-2-yl-ethyl, or morpholin-2-yl-ethyl; or
Y is NHR₂, wherein R₂ is H, C₁₋₂₀ alkyl such as straight chain or branched chain aliphatic residues as defined above, C₃₋₆ cycloalkyl, substituted C₃₋₆ cycloalkyl wherein the substituent is C₁₋₂ alkyl such as methyl or ethyl or trifluoroalkyl such as trifluoromethyl or halogen such as fluorine, chlorine, bromine or R₂ is aryl such as unsubstituted or substituted phenyl wherein the substituent is C₁₋₃ alkyl, C₁₋₃ alkoxy, amino, or halogen; heteroaryl such as pyridyl, pyrimidinyl, quinolinyl, isoquinolinyl, furyl, oxazolinyl, thiazolyl, or thiadiazolyl, all of which heteroaryl residues may be further substituted or unsubstituted, wherein the substituent is methyl or ethyl;
or R₂ is an amino acid residue derived from one of the 20 naturally occurring amino acids viz. alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, or the optically active isomers thereof, or the racemic mixtures thereof;
R₅ is H, C₁₋₅ alkyl, C₁₋₅ alkoxy, amino, C₁₋₅ alkylamino such as-NHCH₃, N(CH₃)₂, and the like; or acylamino such as —NHCOCH₃, —NHCOC(CH₃)₃, and the like;
Q is —N—, —C(R8)— (R₈ being H, F, Cl, bromo, methoxy, C₁₋₄ alkyl, or unsubstituted or substituted C₁₋₄ alkoxy, wherein when the alkoxy is substituted it is substituted by one or more halogen atoms such as F, Cl, or Br), or when Q is CH and the nitrogen atom to which R₁ is linked forms an optionally substituted 5-, 6- or 7-membered ring with the carbon atom of Q, the ring optionally containing one or more hetero atoms selected from nitrogen, oxygen or sulfur atoms, said heteroatom(s) represented by T, preferably R₁ is CH₂CH₂—, CH₂T—, CH₂CH₂CH₂—, CH₂CH₂T—, CH₂TCH₂—, TCH₂T—, TCH₂CH₂CH₂CH₂— CH₂CH₂CH₂T—, CH₂TCH₂CH₂—, or TCH₂CH₂T— where T represents NH, O, or S. If the ring is substituted, the substituent is as defined above for R₁. This 5- to 7-membered ring may be substituted with 1 or 2 of the same substituents as those defined above for R₁, preferably by one C₁-C₅ alkyl group.

### X is OR₄

wherein R₄ is hydrogen, C₁-C₂₀ alkyl as hereinbefore defined, glycosyl, aralkyl such as benzyl; or C₁-C₆ alkanoyl such as acetyl, propionyl, pivaloyl, stearoyl, or nonadecanoyl or aminoalkanoyl such as aminoacetyl, aminopropionyl and the like or an amino acid residue derived from one of the 20 naturally occurring amino acids viz. alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, or the optically active isomers thereof, or the racemic mixtures thereof; or R₄ is 1-aminocyclohexylcarbonyl or COOR₁₁ wherein R₁₁ is as hereinbefore defined or R₄ is —(CH₂)ₙ—CHR₁₀—OCOOR₁₁ where R₁₀ and R₁₁ are as hereinbefore defined, or R₄ is C₆H₁₁O₆, PO₂(CH₃)H, PO₃H₂, PO₂(OCH₃)H or SO₃H thus giving respectively the gluconic acid, phosphonic acid, phosphoric acid and sulfonic acid ester derivatives of the compounds;
or X is NR₆R₇, wherein R₆ is H, C₁₋₂₀ alkyl as hereinbefore defined, C₃₋₆ cycloalkyl, aralkyl such as benzyl, phenethyl, or phenylpropyl; C₁₋₂₀ alkanoyl such as COCH₃, COCH₂CH₃, or COC(CH₃)₃, or C₁₋₂₀ alkoxycarbonyl such as COOCH₃, COOCH₂CH₃, or COOC(CH₃)₃; aralkyloxycarbonyl such as benzyloxycarbonyl, or amino(C₁₋₂₀)alkanoyl such as aminoacetyl, aminopropionyl and the like, or an amino acid residue derived from one of the 20 naturally occurring amino acids or the optically active isomers thereof, or the racemic mixtures thereof. The amino acid residue is derived from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine. The amino acid residue is derived from a single amino acid or from combinations of amino acids that form dipeptide, tripeptide or polypeptide amino acid unit residues wherein a terminal carboxy group is optionally protected by C₁₋₄ alkyl or aralkyl groups and a terminal amino group is optionally protected by a ^{t}-Boc (teritarybutyloxycarbonyl), F-Moc (fluorenylmethoxycarbonyl) or Cbz (benzyloxycarbonyl) group or R₆ may also be COOR₁₁ wherein R₁₁ is as hereinbefore defined or R₆ is C₆H₁₁O₆ thus giving the gluconic acid ester derivative of the compounds.
R₇ is H, C₁₋₆ alkyl as hereinbefore defined, C₃₋₆ cycloalkyl, aralkyl such as benzyl, phenethyl, or phenylpropyl; C₁₋₆ alkanoyl such as COCH₃, COCH₂CH₃, COC(CH₃)₃, aralkyloxycarbonyl such as benzyloxycarbonyl or amino (C₁₋₂₀)alkanoyl such as aminoacetyl, aminopropionyl, etc.; or an amino acid residue derived from one of the 20 naturally occurring amino acids or the optically active isomers thereof, or the racemic mixtures thereof. The amino acid residue is derived from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine. The amino acid residue is derived from a single amino acid or from combinations of amino acids that form dipeptide, tripeptide or polypeptide amino acid unit residues, wherein a terminal carboxy group is optionally protected by C₁₋₄ alkyl or aralkyl groups and a terminal amino group is optionally protected by a ^{t}-Boc (teritarybutyloxycarbonyl), F-Moc (fluorenylmethoxycarbonyl) or Cbz (benzyloxycarbonyl) group or
R₇ may be C₆H₁₁O₆ thus giving the gluconic acid ester derivative of the compounds. R₈/R₈′ are substituents at the 3/3-position of the piperidino ring and are the same or different and represent H, C₁₋₆ alkyl, substituted C₁₋₆ alkyl wherein the substituent is amino, hydroxy, halogen such as one or more fluorine, chlorine, or bromine atoms; alkylamino, or aralkyl such as benzyl.
R₉ is a substituent at the 4-position or 5-position of the piperidino ring and represents H, C₁₋₆ alkyl, C₁₋₅ alkylamino, C₁₋₃ dialkylamino or aryl, aralkyl such as benzyl or phenethyl or a trihaloalkyl such as trifluoromethyl.

In some other embodiments, the antimicrobial agent is one or more of the following:

S-(-)-9-Fluoro-8-(4-hydroxy-piperidin-1-yl)-5-methyl-6,7-dihydro-1-oxo-1H, 5H-benzo [i,j] quinolizine-2-carboxylic acid L-arginine salt tetrahydrate;

2′S, 5S-(-)-9-Fluoro-8-(4-alaninyloxy-piperidin-1-yl)-5-methyl-6,7-dihydro-1-oxo-1H, 5H-benzo [i,j] quinolizine-2-carboxylic acid methane sulfonic acid salt;

(RS)-1-Cyclopropyl-6-fluoro-8-methoxy-7-(4-amino-3,3-dimethyl piperidin-1 -yl)-1,4-dihydro-4-oxo-quinoline-3-carboxylic acid hydrochloride;

(S)-1-Cyclopropyl-6-fluoro-8-methoxy-7-(4-amino-3,3-dimethyl piperidin-1-yl)-1,4-dihydro-4-oxo-quinoline-3-carboxylic acid hydrochloride monohydrate;

(RS)-1-Cyclopropyl-6-fluoro-8-methyl-7-(4-amino-3,3-dimethyl piperidin-1-yl)-1,4-dihydro-4-oxo-quinoline-3-carboxylic acid;

(S)-1-Cyclopropyl-6-fluoro-8-methyl-7-(4-amino-3,3-dimethyl piperidin-1-yl)-1,4-dihydro-4-oxo-quinoline-3-carboxylic acid hydrochloride;

trans-1-Cyclopropyl-6-fluoro-8-methyl-7-(4-hydroxy-3 -methyl piperidin-1-yl)-1,4-dihydro-4-oxo-quinoline-3-carboxylic acid;

It must be understood that the various compounds or agents, described herein generically or specifically, including the antimicrobial agents, antibacterial agents, antifungal agents, and the β-lactam compounds may used in their generally available forms or modified forms, including in a pharmaceutically acceptable forms including, without limitation, salts, prodrugs, esters, ethers, hydrates, metabolites, polymorphs, solvates, complexes, enantiomers, adducts etc.

It must be understood that the invention is not limited by or to any particular antimicrobial agent or β-lactam compound. Rather, the invention has general applicability to a wide variety of antimicrobial agents or β-lactam compounds. In general, the antimicrobial agents, which may be the subject of the invention may also be found in a number of patents and published applications, including U.S. Pat. Nos. 7,626,032; 7,538,221; 7,405,228; 7,393,957; 7,247,642; 7,164,023; 7,132,541; 6,964,966; 6,878,713; 6,753,333; 6,750,224; 6,664,267; 6,608,078; 6,514,986; 4,638,067; 4,665,079; 4,822,801; 5,097,032; 5,051,509; 5,607,942; 5,677,316; 4,777,175; 6,121,285; 6,329,391; 4,874,764; 4,935,420; 5,859,026; 6,121,285; 5,668,286; 5,574,055; 6,358,942; 5,688,792; 6,387,896; 5,977,373; 5,910,504; 5,547,950; 5,700,799; published PCT Application Nos. WO 96/13502; WO 99/24428; WO 98/58923; WO 1993-JP 1925; WO 01/44212; WO 02/06278; WO 00/21960; WO 98/54161; WO 01/58885; WO 01/09107 and WO 00/27830; the disclosures of which are incorporated herein by reference in their entireties as if fully rewritten herein.

A person of skills in the art would appreciate that one way to contacting the microorganism with efflux pump inhibitor may be to position or apply them in such a way that they are in direct or indirect contact with each other, either completely or partially. Yet another way to contacting the microorganism with efflux pump inhibitor could be through surface application of the efflux pump inhibitor at the desired surface or administration into the body of the subject, where the microorganism is likely to be present, in such a way that the efflux pump is likely to come into contact with microorganism, completely or partially. It is preferred that at least a part of the microorganism comes in contact with the efflux pump inhibitor.

In some other embodiments, in methods according to this invention, the efflux pump inhibitor, alone or in combination one or more antimicrobial agents is administered by any appropriate method, which serves to deliver the efflux pump inhibitor and/or antimicrobial agent to the site of the infection. The method of administration can vary depending on various factors, such as for example, the components of the pharmaceutical composition, the site of the potential or actual bacterial infection, the microorganism involved, severity infection, age and physical condition of the subject. Some non-limiting examples of administering the composition to a subject according to this invention include oral, intravenous, topical, intrarespiratory, intraperitoneal, intramuscular, parenteral, sublingual, transdermal, intranasal, aerosol, intraocular, intratracheal, intrarectal, vaginal, gene gun, dermal patch, eye drop, ear drop or mouthwash

The efflux pump inhibitors and/or one or more antimicrobial agent can be administered in a single dosage form or separate dosage forms. Typical, non-limiting examples of dosage forms include solid, semi-solid, liquid and aerosol dosage forms, such as, e.g., tablets, capsules, powders, liquids, suspensions, suppositories, aerosols or the like.

The pharmaceutical compositions according to this invention may include one or more of pharmaceutically acceptable carriers or excipients or the like, Typical, non-limiting examples of such carriers or excipient include mannitol, lactose, starch, magnesium stearate, sodium saccharine, talcum, cellulose, sodium crosscarmellose, glucose, gelatin, sucrose, magnesium carbonate, wetting agents, emulsifying agents, solubilizing agents, pH buffering agents, lubricants, stabilizing agents, binding agents etc.

The pharmaceutical compositions and method disclosed herein are particularly effective against microorganisms previously considered to have limited effectiveness against one or more of the antimicrobial agents. Some non-limiting examples of such organism known to have developed resistance to various antimicrobial agents include E. coli, Pseudomonas aeruginosa, Staphylococcus aureas, Candida albicans etc.

Examples of bacterial infections which can be treated and/or prevented using the methods and/or the pharmaceutical compositions according to this invention include, without limitation, E. coli infections (e.g. urinary tract), Yersinia pestis (pneumonic plague), staphyloccal infection, streptococcal infection, mycobacteria infection, bacterial pneumonia, snigella dysentery, serrate infection, candida infection, cryptococcal infection, methicillin resistant staphylococcus aureus, anthrax, tuberculosis or those caused by Pseudomonas aeruginosa etc.

Examples of fungal infections which can be treated and/or prevented using the methods and/or the pharmaceutical compositions according to this invention include, without limitation thrush, candidiasis, cryptococcosis, histoplasmosis, blastomycosis, aspergillosis, coccidioidomycosis, paracoccidiomycosis, sporotrichosis, zygomycosis, chromoblastomycosis, lobomycosis, mycetoma, onychomycosis, piedra pityriasis versicolor, tinea barbae, tinea capitis, tinea corporis, tinea cruris, tinea favosa, tinea nigra, tinea pedis, otomycosis, phaeohyphomycosis, or rhinosporidiosis. Yeast infections can also be treated and prevented.

The methods and/or compositions according this invention are useful in treating infection caused by Pseudomonas aeruginosa, as well as methicillin resistant Staphylococcus aureus MRSA, which is one of major causative organisms of nosocomial infections. Since these bacteria have multidrug resistance, the treatment of these bacterial infections is difficult, presenting a serious problem in clinical settings. These bacteria acquire drug resistance by drug efflux pump. This pump uses energy to actively transport and discharge drug that has entered inside of the bacteria. Since the efflux pump of Pseudomonas aeruginosa can cause efflux/discharge a variety of antibiotics with different structures, Pseudomonas aeruginosa is resistant to a variety of drugs.

The methods and/or compositions according this invention are particularly useful for pathogenic bacterial species such as Pseudomonas aeruginosa, which is intrinsically resistant to many of the commonly used antibacterial agents. Pseudomonas aeruginosa is gram-negative bacteria with two membranes, outer membrane and inner membrane. In order for drug to be discharged, the drug must be actively transported via these two membranes. The drug efflux pumps are classified into several families. Among them, pumps of RND (resistance nodulation division) family consist of three subunits. Pseudomonas aeruginosa has a plurality of RND pumps. Among them, the major pump is MexAB-OprM pump. Exposing this bacterium to an efflux pump inhibitor can significantly slow the export of an antibacterial agent from the interior of the cell or the export of siderophores. Therefore, if another antibacterial agent is administered in conjunction with the efflux pump inhibitor, the antibacterial agent, which would otherwise be maintained at a very low intracellular concentration by the export process, can accumulate to a concentration, which will inhibit the growth of the bacterial cells. This growth inhibition can be due to either bacteriostatic or bactericidal activity, depending on the specific antibacterial agent used. While P. aeruginosa is an example of an appropriate bacterium, other bacterial and microbial species may contain similar broad substrate pumps, which actively export a variety of antimicrobial agents, and thus can also be appropriate targets.

It will be readily apparent to one skilled in the art that varying substitutions and modifications may be made to the invention disclosed herein without departing from the scope and spirit of the invention. For example, those skilled in the art will recognize that the invention may be practiced using a variety of different compounds within the described generic descriptions.

### EXAMPLES

The following examples illustrate the embodiments of the invention that are presently best known. However, it is to be understood that the following are only exemplary or illustrative of the application of the principles of the present invention. Numerous modifications and alternative compositions, methods, and systems may be devised by those skilled in the art without departing from the spirit and scope of the present invention. The appended claims are intended to cover such modifications and arrangements. Thus, while the present invention has been described above with particularity, the following examples provide further detail in connection with what are presently deemed to be the most practical and preferred embodiments of the invention.

### Example 1

Phe-Arg-beta-naphthylamide (PAN, MC-207110) is reported to inhibit MDR RND transporters in Gram negatives and particularly in P. aeruginosa. We have observed potentiation in the activity of various antimicrobial agents in two clinical isolates, P. aeruginosa 23587 and P. aeruginosa 2301, which express MDR efflux based resistance, in the presence of PAN (Table 1). Synergistic enhancement in the activity of S-(-)-9-fluoro-8-(4-hydroxy-piperidin-1-yl)-5-methyl-6,7-dihydro-1-oxo-1H,5H-benzo [i,j ]quinolizine-2-carboxylic acid L-arginine salt tetrahydrate, azithromycin, and linezolid was noted in the presence of PAN, indicating the detrimental role of efflux pump in these strains. As expected, there was no change in the activity of colistin since generally, it is not reported to be a substrate of efflux, and moreover its target site happens to be the cell surface. Thus in addition to other antimicrobial agents, potentiation of activity of azithromycin—a known substrate of RND pump demonstrates that PAN acts as an efflux pump inhibitor in these strains and the strains employed in the study express MDR RND pumps.

TABLE 1
Restoration of activity of various antimicrobial agents by efflux pump
inhibitor PAN in MDR strains

| | | Diameter of zone of inhibition (m.m.) | | | |
|---|---|---|---|---|---|
| | | P. aeruginosa | | | |
| | Conc. | 23587 | | P. aeruginosa 2301 | |
| | (μg/ | | +PAN | | +PAN |
| Antimicrobial agent | well) | Control | (20 μg/ml) | Control | (20 μg/ml) |
| S-(−)-9-Fluoro-8- | 4 | Nil | 30 | 15.5 | 25 |
| (4-hydroxy-piperidin- | 8 | 12 | 34 | 20 | 32 |
| 1-yl)-5-methyl-6,7- | 16 | 15 | 35 | 23.5 | 38 |
| dihydro-1-oxo-1H,5H- | | | | | |
| benzo [i,j] quinolizine- | | | | | |
| 2-carboxylic acid | | | | | |
| L-arginine salt | | | | | |
| tetrahydrate. | | | | | |
| Azithromycin | 12.5 | SLI | 16 | SLI | 25 |
| | 25 | 19H | 23.5 | SLI | 28 |
| | 50 | 23H | 27 | 13.5H | 32 |
| Ciprofloxacin | 1 | 10 | Nil | 20 | 32 |
| | 2 | 15 | 21 | 25 | 38 |
| | 4 | 23 | 26 | 28 | 42 |
| Colistin | 3.12 | 12.5 | 30 | 12 | 12 |
| | 6.25 | 14 | 14 | 14 | 14 |
| | 12.5 | 16.5 | 16.5 | 16 | 16 |

H: Hazy zone of inhibitions denoting partial growth inhibition;
PAN: Phe-Arg-beta-naphthylamide (MC-207110),
SLI: Slight indicative activity

### Example 2

Table 2 shows results of activity of various antimicrobial agents in the presence of reserpine and sodium azide. Reserpine is a well-characterized inhibitor of ABC transporter based efflux pump but has been reported to have little activity against RND family pumps.

Therefore, as expected no change in the activity of antimicrobial agents including azithromycin was observed in presence of reserpine, suggesting that the strains employed do not posses ABC transporters as efflux pumps and therefore diminished activity of antimicrobial agents is not attributable to ABC transporter pumps.

Since RND efflux pumps operate by utilizing energy in the form of ATP, metabolic inhibitor, sodium azide brings about MDR RND pump inhibition by causing energy deprivation. The addition of sodium azide potentiated activity of various antimicrobial agents such as S-(-)-9-Fluoro-8-(4-hydroxy-piperidin-1-yl)-5-methyl-6,7-dihydro-1-oxo-1H, 5H-benzo [i,j] quinolizine-2-carboxylic acid L-arginine salt tetrahydrate; azithromycin and ethidium bromide, which are known substrates of MDR RND pumps indicating that ATP dependent RND pumps operating in these strains play a critical role in resistance to multiple antimicrobial agents. Thus, efflux inhibition by PAN and sodium azide and non inhibition by reserpine establishes the dominant role of MDR RND in these clinical isolates thereby imparting a very high degree of resistance to most antimicrobial agents.

TABLE 2
Restoration of activity of various antimicrobial agents by energy
deprivation based efflux pump inhibition by Sodium azide in Reserpine
multi-drug resistant P. aeruginosa

| | | Diameter of zone of inhibition | | |
|---|---|---|---|---|
| | | (m.m.) | | |
| | | P. aeruginosa (23587) | | |
| | Conc. | | +sodium | |
| | (μg/ | | azide | +Reserpine |
| Antimicrobial agent | well) | Control | (10 μg/ml) | (25 μg/ml) |
| S-(−)-9-Fluoro-8-(4- | 8 | Nil | 12 | Nil |
| hydroxy-piperidin-1-yl)-5- | 16 | 11 | 15 | 11 |
| methyl-6,7-dihydro-1-oxo- | 32 | 15 | 20 | 15 |
| 1H,5H-benzo [i,j] | | | | |
| quinolizine-2-carboxylic | | | | |
| acid L-arginine salt | | | | |
| tetrahydrate. | | | | |
| Azithromycin | 50 | 12H | 21H | 12H |
| | 100 | 20H | 24H | 20H |
| | 200 | 24H | 30 | 24H |
| Ethidium Bromide | 50 | Nil | 10H | Nil |
| | 100 | Nil | 13H | Nil |
| | 200 | Nil | 16H | Nil |
| Colistin | 3.12 | 11.5 | 11.5 | 11.5 |
| | 6.25 | 14 | 14 | 14 |
| | 12.5 | 16 | 16 | 16 |

H: Hazy zone of inhibitions denoting partial growth inhibition;

### Example 3

Table 3 shows activity of various antimicrobial agents in the presence of β-lactam compounds (ceftazidime and cefepime). In a most surprising and unexpected manner, we found significant potentiation in the activity of S-(-)-9-Fluoro-8-(4-hydroxy-piperidin-1-yl)-5-methyl-6,7-dihydro-1-oxo-1H, 5H-benzo [i,j] quinolizine-2-carboxylic acid L-arginine salt tetrahydrate and azithromycin in the presence of cefepime and ceftazidime. Potentiation of activity of azithromycin and other antimicrobial agents, which are known RND pump substrates in these strains, demonstrates that cefepime and ceftazidime are inhibit RND pumps thereby increasing the intracellular uptake of these antimicrobial agents. Thus PAN, sodium azide, cefepime and ceftazidime showed common synergistic profile suggesting a direct role of β-lactam compounds in the inhibition of RND pumps. The finding is highly surprising since the requirement of β-lactam as efflux pump inhibitor is ⅛ or 1/16 of their MIC, typical of an efflux pump inhibitor.

TABLE 3
Restoration of activity of various antimicrobial agents by using β-lactam
compounds (cefepime and ceftazidime) as efflux pump inhibitors in multi-
drug resistant P. aeruginosa

| | | Diameter of zone of inhibition (m.m.) | | |
|---|---|---|---|---|
| | Conc. | P. aeruginosa 23587 | | |
| | (μg/ | | + Cefepime | + Ceftazidime |
| Antimicrobial agent | Well) | Control | (1 μg/ml) | (1 μg/ml) |
| S-(−)-9-Fluoro-8-(4- | 8 | 9 | 12 | 20 |
| hydroxy-piperidin-1- | 16 | 14.5 | 18 | 25 |
| yl)-5-methyl-6,7- | 32 | 19.5 | 24 | 33 |
| dihydro-1-oxo-1H,5H- | | | | |
| benzo [i,j] quinolizine- | | | | |
| 2-carboxylic acid | | | | |
| L-arginine salt | | | | |
| tetrahydrate. | | | | |
| Azithromycin | 50 | 12h | 24H | 32 |
| | 100 | 20H | 28H | 36 |
| | 200 | 24H | 30H | 42 |
| Ciprofloxacin | 1 | 10 | 13 | 17 |
| | 2 | 15 | 20.5 | 24 |
| | 4 | 23 | 27 | 30 |
| Colistin | 3.12 | 11.5 | 11 | 11 |
| | 6.25 | 14 | 14 | 14 |
| | 12.5 | 16.5 | 16 | 16 |

H: Hazy zone of inhibitions denoting partial growth inhibition;

### Example 4

Table 4 results of efflux pump inhibition based cidal synergy between S-(-)-9-Fluoro-8-(4-hydroxy-piperidin-1-yl)-5-methyl-6,7-dihydro-1-oxo-1H, 5H-benzo [i,j] quinolizine-2-carboxylic acid L-arginine salt tetrahydrate nd ceftazidime. Potent cidal synergy is observed between S-(-)-9-Fluoro-8-(4-hydroxy-piperidin-1-yl)-5-methyl-6,7-dihydro-1-oxo-1H, 5H-benzo [i,j] quinolizine-2-carboxylic acid L-arginine salt tetrahydrate (concentration, 8 μg/ml) and ceftazidime (concentration, 8 and 16 μg/ml). These concentrations are at least 4 time slower than their MIC. While both the agents are ineffective individually, their combination brings about >3 log (>99.99%) reduction at 24 and 48 h. Thus the efflux pump inhibition based synergy is not just growth inhibitory but also, most importantly, imparts extensive killing of the pathogen.

TABLE 4
Enhanced killing of MDR P. aeruginosa, mediated through improved uptake of
antimicrobial agent in the presence of efflux pump inhibitor ceftazidime

| | | Log CFU/ml | | |
|---|---|---|---|---|
| | Concentration | viable count at | | |
| Antimicrobial agent | (μg/ml) | 0 h | 24 h | 48 h |
| Drug free Control | Nil | 6.11 | 8.7 | 9.2 |
| Ceftazidime | 8 | 6.11 | 8.64 | 9.3 |
| Ceftazidime | 16 | 6.11 | 5.30 | 9.2 |
| S-(−)-9-Fluoro-8-(4-hydroxy- | 8 | 6.11 | 8.64 | 9.4 |
| piperidin-1-yl)-5-methyl-6,7- | 16 | 6.11 | 4.30 | <3.0 |
| dihydro-1-oxo-1H,5H-benzo [i,j] | | | | |
| quinolizine-2-carboxylic acid L- | | | | |
| arginine salt tetrahydrate | | | | |
| Ceftazidime + | Ceftazidime (8) + | 6.11 | <3.0 | <3.0 |
| S-(−)-9-Fluoro-8-(4-hydroxy- | S-(−)-9-Fluoro-8-(4-hydroxy- | | | |
| piperidin-1-yl)-5-methyl-6,7- | piperidin-1-yl)-5-methyl-6,7- | | | |
| dihydro-1-oxo-1H,5H-benzo [i,j] | dihydro-1-oxo-1H,5H-benzo [i,j] | | | |
| quinolizine-2-carboxylic acid L- | quinolizine-2-carboxylic acid L- | | | |
| arginine salt tetrahydrate. | arginine salt tetrahydrate (8) | | | |
| | Ceftazidime (16) + | 6.11 | <3.0 | <3.0 |
| | S-(−)-9-Fluoro-8-(4-hydroxy- | | | |
| | piperidin-1-yl)-5-methyl-6,7- | | | |
| | dihydro-1-oxo-1H,5H-benzo [i,j] | | | |
| | quinolizine-2-carboxylic acid L- | | | |
| | arginine salt tetrahydrate (8) | | | |

### Example 5

Table 5 demonstrates the efflux based synergy between β-lactam compound and S-(-) -9-Fluoro-8-(4-hydroxy-piperidin-1-yl)-5-methyl-6,7-dihydro-1-oxo-1H, 5H-benzo [i,j] quinolizine-2-carboxylic acid L-arginine salt tetrahydrate' in animal model of infections. S-(-)-9-Fluoro-8-(4-hydroxy-piperidin-1-yl)-5-methyl-6,7-dihydro-1-oxo-1H, 5H-benzo [i,j] quinolizine-2-carboxylic acid L-arginine salt tetrahydrate and ceftazidime individually at 50 and 300 mg/kg dose could not protect animals infected with highly resistant P. aeruginosa strains (S-(-)-9-Fluoro-8-(4-hydroxy-piperidin-1-yl)-5-methyl-6,7-dihydro-1-oxo-1H, 5H-benzo [i,j] quinolizine-2-carboxylic acid L-arginine salt tetrahydrate MIC, 16 μg/ml and ceftazidime MIC, >32 μg/ml). Surprisingly, a combination of S-(-)-9-Fluoro-8-(4-hydroxy-piperidin-1-yl)-5-methyl-6,7-dihydro-1-oxo-1H, 5H-benzo [i,j] quinolizine-2-carboxylic acid L-arginine salt tetrahydrate at 50 mg/kg and ceftazidime at 75 mg/kg protected 100% of animals. Thus, typical of an efflux pump inhibitor, ceftazidime, at a several fold lower than its own effective dose, brings about 100% survival in combination with sub-effective doses of S-(-)-9-Fluoro-8-(4-hydroxy-piperidin-1-yl)-5-methyl-6,7-dihydro-1-oxo-1H, 5H-benzo [i,j] quinolizine-2-carboxylic acid L-arginine salt tetrahydrate.

TABLE 5
Synergistic therapeutic outcome facilitated by combination of S-(−)-9-Fluoro-8-(4-
hydroxy-piperidin-1-yl)-5-methyl-6,7-dihydro-1-oxo-1H,5H-benzo [i,j] quinolizine-2-
carboxylic acid L-arginine salt tetrahydrate with efflux pump inhibitor Ceftazidime in
systemic infection caused by MDR clinical isolate of P. aeruginosa 2301 in mice

| | Dose (mg/kg) | Treatment | % |
|---|---|---|---|
| Antimicrobial agent | BID × 1 day | Route | survival |
| S-(−)-9-Fluoro-8-(4-hydroxy- | 50 | Subcutaneous | 0 |
| piperidin-1-yl)-5-methyl-6,7- | 75 | Subcutaneous | 33.3 |
| dihydro-1-oxo-1H,5H-benzo [i,j] | 100 | Subcutaneous | 100 |
| quinolizine-2-carboxylic acid L- | | | |
| arginine salt tetrahydrate | | | |
| Ceftazidime | 300 | Subcutaneous | 16.6 |
| Ceftazidime + | Ceftazidime (75) + | Subcutaneous | 50 |
| S-(−)-9-Fluoro-8-(4-hydroxy- | S-(−)-9-Fluoro-8-(4-hydroxy- | | |
| piperidin-1-yl)-5-methyl-6,7- | piperidin-1-yl)-5-methyl-6,7- | | |
| dihydro-1-oxo-1H,5H-benzo [i,j] | dihydro-1-oxo-1H,5H-benzo | | |
| quinolizine-2-carboxylic acid L- | [i,j] quinolizine-2- | | |
| arginine salt tetrahydrate | carboxylic acid L-arginine | | |
| | salt tetrahydrate (25) | | |
| Ceftazidime + | Ceftazidime (75) + | Subcutaneous | 100 |
| S-(−)-9-Fluoro-8-(4-hydroxy- | S-(−)-9-Fluoro-8-(4-hydroxy- | | |
| piperidin-1-yl)-5-methyl-6,7- | piperidin-1-yl)-5-methyl-6,7- | | |
| dihydro-1-oxo-1H,5H-benzo [i,j] | dihydro-1-oxo-1H,5H-benzo | | |
| quinolizine-2-carboxylic acid L- | [i,j] quinolizine-2- | | |
| arginine salt tetrahydrate | carboxylic acid L-arginine | | |
| | salt tetrahydrate (50) | | |

Thus, considering the data provided in above examples, we have shown that β-lactam compounds, such as cefepime and ceftazidime, indeed inhibit MDR efflux, particularly the RND pumps in gram negatives thereby increasing the intracellular concentrations of S-(-)-9-Fluoro-8-(4-hydroxy-piperidin-1-yl)-5-methyl-6,7-dihydro-1-oxo-1H, 5H-benzo [i,j] quinolizine-2-carboxylic acid L-arginine salt tetrahydrate, azithromycin and various other antimicrobial agents in the vicinity of their respective targets. We have also validated the above mentioned concept in animal model of infection and shown that the in vitro inhibition of efflux pump by β-lactam very well translates into a potent bactericidal synergy in vivo with S-(-)-9-Fluoro-8-(4-hydroxy-piperidin-1-yl)-5-methyl-6,7-dihydro-1-oxo-1H, 5H-benzo [i,j] quinolizine-2-carboxylic acid L-arginine salt tetrahydrate leading to 100% protection of animals from severe Pseudomonas infection.

We have also demonstrated that clinical use of S-(-)-9-Fluoro-8-(4-hydroxy-piperidin-1-yl)-5-methyl-6,7-dihydro-1-oxo-1H, 5H-benzo [i,j] quinolizine-2-carboxylic acid L-arginine salt tetrahydrate and a β-lactam compound combination, such as cefepime and ceftazidime, can provide a much needed armamentarium against most difficult to treat gram negative infections and particularly infections caused by highly resistant MDR strains of P. aeruginosa.

### Example 6

Table 6 gives results on restoration of activity of various antimicrobial agents when used in combination with efflux pump inhibitors (cefepime and ceftazidime) in MDR P. aeruginosa. Table 3 shows activity of various antimicrobial agents in the presence of β-lactam compounds (ceftazidime and cefepime). In a most surprising and unexpected manner, we found significant potentiation in the activity of S-(-)-9-Fluoro-8-(4-hydroxy-piperidin-1-yl)-5-methyl-6,7-dihydro-1-oxo-1H, 5H-benzo [i,j] quinolizine-2-carboxylic acid L-arginine salt tetrahydrate and azithromycin in the presence of cefepime and ceftazidime. Potentiation of activity of azithromycin and other antimicrobial agents, which are known RND pump substrates in these strains, demonstrates that cefepime and ceftazidime are inhibit modulate RND pumps thereby increasing the intracellular uptake of these antimicrobial agents. It is also likely that ceftazidime and cefepime interacts with outer membrane porins and acts as a porin modulator. Bacalum et al. have shown that ceftazidime binds to outer membrane porins with high affinity (Bacalum et al. Romanian. J. Biophys., 19, 105-116, 2009).

TABLE 6
Restoration of activity of various antimicrobial agents including when used
in combination with efflux pump inhibitors (cefepime and ceftazidime)

| | | Diameter of zone of inhibition (m.m.) | | |
|---|---|---|---|---|
| | Conc. | P. aeruginosa 2301 | | |
| | (μg/ | | +Cefepime | +Ceftazidime |
| Antimicrobial agents | Well) | Control | (2 μg/ml) | (0.5 μg/ml) |
| S-(−)-9-Fluoro-8-(4- | 8 | 9 | 12 | 20 |
| hydroxy-piperidin-1- | 16 | 14.5 | 18 | 25 |
| yl)-5-methyl-6,7- | 32 | 19.5 | 24 | 33 |
| dihydro-1-oxo-1H,5H- | | | | |
| benzo [i,j] quinolizine- | | | | |
| 2-carboxylic acid | | | | |
| L-arginine salt | | | | |
| tetrahydrate. | | | | |
| Azithromycin | 50 | 12H | 24H | 32H |
| | 100 | 20H | 28H | 36H |
| | 200 | 24H | 30H | 42H |
| Ciprofloxacin | 1 | Nil | 23.5H | SLI |
| | 2 | Nil | 24H | SLI |
| | 4 | Nil | 25H | SLI |
| Colistin | 3.12 | 11.5 | 11 | 11 |
| | 6.25 | 14 | 14 | 14 |
| | 12.5 | 16.5 | 16 | 16 |

H: Hazy zone of inhibitions denoting partial growth inhibition;
SLI: Slight indicative activity

### Example 7

Table 7 gives results on synergistic therapeutic outcome facilitated by combination of quinolone with efflux pump inhibitor Ceftazidime in systemic infection caused by MDR clinical isolate of P. aeruginosa 2301 in mice.

TABLE 7
Synergistic therapeutic outcome facilitated by combination of quinolone with efflux
pump inhibitor Ceftazidime in systemic infection caused by MDR clinical isolate of
P. aeruginosa 2301 in mice

| | Dose (mg/kg) | Treatment | % |
|---|---|---|---|
| Drug/Combination | BID × 1 day | Route | survival |
| 2′S,5S-(−)-9-Fluoro-8-(4- | 50 | Oral | 0 |
| alaninyloxy-piperidin-1-yl)-5- | 75 | Oral | 16.6 |
| methyl-6,7-dihydro-1-oxo-1H, | 100 | Oral | 16.6 |
| 5H-benzo [i,j] quinolizine-2- | | | |
| carboxylic acid methane sulfonic | | | |
| acid salt. | | | |
| Ceftazidime | 100 | Oral | 16.6 |
| Ceftazidime | 200 | Oral | 16.6 |
| Ceftazidime + | Ceftazidime (100) + | Oral | 80 |
| 2′S,5S-(−)-9-Fluoro-8-(4- | 2′S,5S-(−)-9-Fluoro-8-(4- | | |
| alaninyloxy-piperidin-1-yl)-5- | alaninyloxy-piperidin-1-yl)-5- | | |
| methyl-6,7-dihydro-1-oxo-1H, | methyl-6,7-dihydro-1-oxo-1H, | | |
| 5H-benzo [i,j] quinolizine-2- | 5H-benzo [i,j] quinolizine-2- | | |
| carboxylic acid methane sulfonic | carboxylic acid methane | | |
| acid salt. | sulfonic acid salt (50) | | |
| Ceftazidime + | Ceftazidime (100) + | Oral | 90 |
| 2′S,5S-(−)-9-Fluoro-8-(4- | 2′S,5S-(−)-9-Fluoro-8-(4- | | |
| alaninyloxy-piperidin-1-yl)-5- | alaninyloxy-piperidin-1-yl)-5- | | |
| methyl-6,7-dihydro-1-oxo-1H, | methyl-6,7-dihydro-1-oxo-1H, | | |
| 5H-benzo [i,j] quinolizine-2- | 5H-benzo [i,j] quinolizine-2- | | |
| carboxylic acid methane sulfonic | carboxylic acid methane | | |
| acid salt. | sulfonic acid salt (75) | | |
| Ceftazidime + | Ceftazidime (100) + | Oral | 100 |
| 2′S,5S-(−)-9-Fluoro-8-(4- | 2′S,5S-(−)-9-Fluoro-8-(4- | | |
| alaninyloxy-piperidin-1-yl)-5- | alaninyloxy-piperidin-1-yl)-5- | | |
| methyl-6,7-dihydro-1-oxo-1H, | methyl-6,7-dihydro-1-oxo-1H, | | |
| 5H-benzo [i,j] quinolizine-2- | 5H-benzo [i,j] quinolizine-2- | | |
| carboxylic acid methane sulfonic | carboxylic acid methane | | |
| acid salt. (WCK 2349); | sulfonic acid salt (100) | | |

### Example 8

Table 8 shows activity of S-(-)-9-Fluoro-8-(4-hydroxy-piperidin-1-yl)-5-methyl-6,7-dihydro-1-oxo-1H, 5H-benzo [i,j] quinolizine-2-carboxylic acid L-arginine salt tetrahydrate in the presence of ceftazidime under the challenge of high density highly resistant Pseudomonas strain. In a most surprising manner, the combination of ceftazidime and S-(-)-9-Fluoro-8-(4-hydroxy-piperidin-1-yl)-5-methyl-6,7-dihydro-1-oxo-1H, 5H-benzo [i,j] quinolizine-2-carboxylic acid L-arginine salt tetrahydrate brought about more than 6 log reduction in the bacterial count as compared to individual effects. Moreover in a very unusual and surprising manner this tremendous bactericidal effect at high cell density inoculum is taking place at concentrations much below than their individual inhibitory concentrations. Potentiation of S-(-)-9-Fluoro-8-(4-hydroxy-piperidin-1-yl)-5-methyl-6,7-dihydro-1-oxo-1H, 5H-benzo [i,j] quinolizine-2-carboxylic acid L-arginine salt tetrahydrate which are known RND pump substrates in these strains, demonstrates that ceftazidime inhibit/modulates RND pumps thereby increasing the intracellular uptake of these antimicrobial agents. It is also likely that ceftazidime interacts with outer membrane porins and acts as a porin modulator. Bacalum et al. have shown that ceftazidime binds to outer membrane porins with high affinity (Bacalum et al. Romanian. J. Biophys., 19, 105-116, 2009).

TABLE 8
Efflux pump inhibition mediated potentiation of WCK 771 by Ceftazidime
against quinolone and cephalosporin resistant strain of P. aeruginosa
23587

| | Survival of High Density | |
|---|---|---|
| | (2-5 × 10⁹ CFU/ml) | |
| | Inoculation | |
| | Drug Concentration in MHA medium | |
| | | CFU/ml |
| Drug/Combination | Concentration (mcg/ml) | at 48 h |
| Drug Free Control | 0 | >5 × 10⁹ |
| S-(−)-9-Fluoro-8-(4- | 32 | >5 × 10⁹ |
| hydroxy-piperidin-1-yl)- | | |
| 5-methyl-6,7-dihydro-1- | | |
| oxo-1H,5H-benzo | | |
| [i,j] quinolizine- | | |
| 2-carboxylic acid | | |
| L-arginine salt | | |
| tetrahydrate | | |
| Ceftazidime | 64 | >5 × 10⁹ |
| S-(−)-9-Fluoro-8-(4- | S-(−)-9-Fluoro-8-(4-hydroxy- | 1 × 10³ |
| hydroxy-piperidin-1-yl)-5- | piperidin-1-yl)-5-methyl-6,7- | |
| methyl-6,7-dihydro-1- | dihydro-1-oxo-1H,5H-benzo | |
| oxo-1H,5H-benzo [i,j] | [i,j] quinolizine-2-carboxylic | |
| quinolizine-2-carboxylic | acid L-arginine salt | |
| acid L-arginine salt | tetrahydrate (8) + | |
| tetrahydrate + | Ceftazidime (4) | |
| Ceftazidime | S-(−)-9-Fluoro-8-(4-hydroxy- | 1.25 × 10² |
| | piperidin-1-yl)-5-methyl-6,7- | |
| | dihydro-1-oxo-1H,5H-benzo | |
| | [i,j] quinolizine-2-carboxylic | |
| | acid L-arginine salt | |
| | tetrahydrate (8) + | |
| | Ceftazidime (8) | |

CFU: Colony forming Units
MHA: Mueller Hinton Agar

### Example 9

Table 9 shows activity of azithromycin and tigecycline in the presence of ceftazidime under the challenge of high density bacteria. In a most surprising manner, the combination of ceftazidime and azithromycin and tigecycline respectively brought about more than 7 log reduction in the bacterial count as compared to individual effects in highly resistant Pseudomonas strain. Moreover in a very unusual and surprising manner this tremendous bactericidal effect is taking place at concentrations much below than their individual inhibitory concentrations. Potentiation of tigecycline and azithromycin which are known RND pump substrates in these strains, demonstrates that ceftazidime inhibit/modulates RND pumps thereby increasing the intracellular uptake of these antimicrobial agents. It is also likely that ceftazidime interacts with outer membrane porins and acts as a porin modulator. Bacalum et al. have shown that ceftazidime binds to outer membrane porins with high affinity (Bacalum et al. Romanian. J. Biophys., 19, 105-116, 2009).

TABLE 9
Efflux pump inhibition mediated potentiation of Azithromycin and
Tigecycline by Ceftazidime against multidrug resistant strain of
P. aeruginosa 2301

Survival of High Density
(2-5 × 10⁹ CFU/ml) Inoculation
Drug Concentration in MHA medium

| Drug combination | Concentration (mcg/ml) | CFU/ml at 48 h |
|---|---|---|
| Drug Free | 0 | >5 × 10⁹ |
| Control | | |
| Azithromycin | 20 | >5 × 10⁹ |
| Tigecycline | 20 | >5 × 10⁹ |
| Ceftazidime | 20 | >5 × 10⁹ |
| Azithromycin + | Azithromycin - 10 | <1.0 × 10² |
| Ceftazidime | Ceftazidime - 10 | |
| | Azithromycin - 20 | <1.0 × 10² |
| | Ceftazidime - 10 | |
| Tigecycline + | Tigecycline - 10 | <1.0 × 10² |
| Ceftazidime | Ceftazidime - 10 | |
| | Tigecycline - 20 | <1.0 × 10² |
| | Ceftazidime - 10 | |

CFU: Colony forming Units
MHA: Mueller Hinton Agar

1. A method of inhibiting efflux pump activity in a microorganism, comprising contacting said microorganism with an effective amount of an efflux-pump inhibitor, wherein said efflux-pump inhibitor is a β-lactam compound.

2. (canceled)

3. A method of treating infection caused by a microorganism in a subject, comprising administering to the subject in need thereof, a therapeutically effective amount of an efflux-pump inhibitor in combination with at least one antimicrobial agent, wherein said efflux-pump inhibitor is a β-lactam compound.

4. A method for prophylactic treatment of a subject, comprising administering to a subject at risk of infection caused by microorganism, a prophylactically effective amount of an efflux-pump inhibitor, wherein said efflux-pump inhibitor is a β-lactam compound.

5. A method for prophylactic treatment of a subject, comprising administering to a subject at risk of infection by microorganism, a prophylactically effective amount of an efflux-pump inhibitor in combination with at least one antimicrobial agent, wherein said efflux-pump inhibitor is a β-lactam compound.

6. A pharmaceutical composition effective for treatment of infection in a subject by a microorganism, comprising an efflux-pump inhibitor in combination with at least one antimicrobial agent, wherein said efflux-pump inhibitor is a β-lactam compound.

7. (canceled)

8. A method or composition according to claim 1, wherein the microorganism at least one microorganism selected from a bacteria, fungi, protozoa, yeast, mold, or mildew.

9. A method or composition according to claim 1, wherein the β-lactam compound is at least one selected from Cefazolin, Cefacetrile, Cefadroxil, Cefalexin, Cefaloglycin, Cefalonium, Cefaloridine, Cefalotin, Cefapirin, Cefatrizine, Cefazedone, Cefazaflur, Cefradine, Cefroxadine, Ceftezole, Cefaclor, Cefamandole, Cefminox, Cefonicid, Ceforanide, Cefotiam, Cefprozil, Cefbuperazone, Cefuroxime, Cefuzonam, cephamycin, cefoxitin, cefotetan, Cefmetazole, carbacephem, Cefixime, Ceftazidime, Ceftriaxone, Cefcapene, Cefdaloxime, Cefdinir, Cefditoren, Cefetamet, Cefmenoxime, Cefodizime, Cefoperazone, Cefotaxime, Cefpimizole, Cefpiramide, Cefpodoxime, Cefsulodin, Cefteram, Ceftibuten, Ceftiolene, Ceftizoxime, oxacephem, Cefepime, Cefozopran, Cefpirome, Cefquinome, Ceftobiprole, Ceftiofur, Cefquinome, Cefovecin, Amoxicillin, Ampicillin, Azlocillin, Carbenicillin, Cloxacillin, Dicloxacillin, Flucloxacillin, Mezlocillin, Meticillin, Nafcillin, Oxacillin, Penicillin, Piperacillin, Ticarcillin, Ertapenem, Doripenem, Imipenem, Meropenem, and Sulopenem, CXA 101, Ceftaroline, Ceftobiprole, Aztreonam.

10. (canceled)

11. (canceled)

12. A method according to claim 3, wherein the antimicrobial agent is at least one selected from an antibiotic agent, antibacterial agent or antifungal agent.

13. A method or a pharmaceutical composition according claim 12, wherein the antibacterial agents is at least one selected from aminoglycoside, oxazolidinone, quinolone, ansamycin, carbacephem, carbapenem, cephalosporin, glycopeptide, macrolide, penicillin, sulfonamide, polypeptide or a tetracycline antibacterial agent.

14. A method or a pharmaceutical composition according to claim 13, wherein the antibacterial agent is at least one aminoglycoside antibacterial agent selected from amikacin, gentamicin, kanamycin, neomycin, netilmicin, streptomycin, tobramycin or paromomycin.

15. A method or a pharmaceutical composition according to claim 13, wherein the antibacterial agent is at least one oxazolidinone antibacterial agent selected from linezolid, ranbezolid, torezolid or radezolid.

16. A method or a pharmaceutical composition according to claim 13, wherein the antibacterial agent is at least one quinolone antibacterial agent selected from cinoxacin, flumequine, nalidixic acid, oxolinic acid, piromidic acid, pipemidic acid, rosoxacin, ciprofloxacin, enoxacin, fleroxacin, lomefloxacin, nadifloxacin, norfloxacin, ofloxacin, pefloxacin, rufloxacin, balufloxacin, gatifloxacin, grepafloxacin, levofloxacin, moxifloxacin, pazufloxacin, sparfloxacin, temafloxacin, tosufloxacin, clinafloxacin, gemifloxacin, sitafloxacin, trovafloxacin, prulifloxacin, garenoxacin, delafloxacin, danofloxacin, difloxacin, enrofloxacin, ibafloxacin, marbofloxacin, orbifloxacin, sarafloxacin, nemonoxacin, finafloxacin, or delafloxacin.

17. A method or pharmaceutical composition according to claim 13, wherein the antibacterial agent is a quinolone compound having general formula (I): 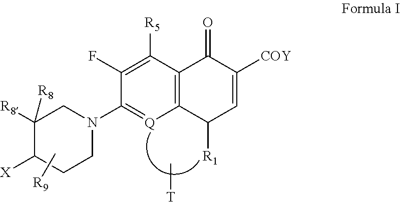
Wherein; R₁ is C₁-₅ alkyl being unsubstituted or substituted with from 1 to 3 fluoro atoms, C₃₋₆ cycloalkyl being unsubstituted or substituted with from 1 to 2 fluoro atoms, or aryl being unsubstituted or substituted with from 1 to 3 fluoro atoms;
or when Q is CH and the nitrogen atom to which R₁ is linked forms an optionally substituted 5-, 6- or 7-membered ring with the carbon atom of Q, the ring optionally containing one or more hetero atoms selected from nitrogen, oxygen or sulfur atoms, said heteroatom(s) represented by T, preferably R₁ is CH₂CH₂—, CH₂T—, CH₂CH₂CH₂—, CH₂CH₂T—, CH₂TCH₂—, TCH₂T—, TCH₂CH₂CH₂CH₂— CH₂CH₂CH₂T—, CH₂TCH₂CH₂—, or TCH₂CH₂T— where T represents NH, O, or S. This 5- to 7-membered ring may be substituted with 1 or 2 of the same substituents as those defined above for R₁, preferably by one C₁-C₅ alkyl group.
Y is OR₃ where
R₃ is hydrogen;
R₃ is C₁-C₂₀ alkyl, such as straight chain or branched chain aliphatic residues such as methyl, ethyl, propyl, isopropyl, butyl, isobutyl, tertiary butyl, pentyl, hexyl or their branched chain isomers;
R₃ is aralkyl such as benzyl, phenethyl, or phenylpropyl;
R₃ is CH₂CH(NH₂)COOH;
R₃ is (CH₂)ₙ—CHR₁₀—OCOR₁₁ or (CH₂)ₙ—CHR₁₀—OCO₂R₁₁ wherein R₁₀ is H, or CH₃; n is 0-3 and R₁₁ is C₁-C₂₀ alkyl as hereinbefore defined, or substituted C₁-C₆ alkyl with substituents such as hydroxy, halogen, amino, or mercapto; or aralkyl such as benzyl, phenethyl, phenylpropyl or R₁₁ is 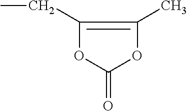
or R₃ is ∀-aminoalkanoyl such as ∀-aminopropionyl or R₃ is alkanoylalkyl group such as acetoxymethyl, acetoxyethyl, pivaloyloxy-methyl, or pivaloyloxyethyl group;
or R₃ is 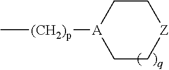
wherein;
A is CH or N, and when A is CH, Z is NH or NCH₃, and when A is N, Z is CH, O, NH, S, or NCH₃ ; p is 0-2; q is 0-2, preferably it is a group such as N-methylpiperidin-4-yl, pyrrolidin-2-yl-ethyl, piperidin-2-yl-ethyl, or morpholin-2-yl-ethyl; or
Y is NHR₂, wherein R₂ is H, C₁-₂₀ alkyl such as straight chain or branched chain aliphatic residues as defined above, C₃₋₆ cycloalkyl, substituted C₃₋₆ cycloalkyl wherein the substituent is C₁₋₂ alkyl such as methyl or ethyl or trifluoroalkyl such as trifluoromethyl or halogen such as fluorine, chlorine, bromine or R₂ is aryl such as unsubstituted or substituted phenyl wherein the substituent is C₁₋₃ alkyl, C₁₋₃ alkoxy, amino, or halogen; heteroaryl such as pyridyl, pyrimidinyl, quinolinyl, isoquinolinyl, furyl, oxazolinyl, thiazolyl, or thiadiazolyl, all of which heteroaryl residues may be further substituted or unsubstituted, wherein the substituent is methyl or ethyl;
or R₂ is an amino acid residue derived from one of the 20 naturally occurring amino acids viz. alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, or the optically active isomers thereof, or the racemic mixtures thereof;
R₅ is H, C₁₋₅ alkyl, C₁₋₅ alkoxy, amino, C₁₋₅ alkylamino such as —NHCH₃, N(CH₃)₂, and the like; or acylamino such as —NHCOCH₃, —NHCOC(CH₃)₃, and the like;
Q is —N—, —C(R₈)— (R₈ being H, F, Cl, bromo, methoxy, C₁₋₄ alkyl, or unsubstituted or substituted C₁₋₄ alkoxy, wherein when the alkoxy is substituted it is substituted by one or more halogen atoms such as F, Cl, or Br), or when Q is CH and the nitrogen atom to which R₁ is linked forms an optionally substituted 5-, 6- or 7-membered ring with the carbon atom of Q, the ring optionally containing one or more hetero atoms selected from nitrogen, oxygen or sulfur atoms, said heteroatom(s) represented by T, preferably R₁ is CH₂CH₂—, CH₂T—, CH₂CH₂CH₂—, CH₂CH₂T—, CH₂TCH₂—, TCH₂T—, TCH₂CH₂CH₂CH₂— CH₂CH₂CH₂T—, CH₂TCH₂CH₂—, or TCH₂CH₂T— where T represents NH, O, or S. If the ring is substituted, the substituent is as defined above for R₁. This 5- to 7-membered ring may be substituted with 1 or 2 of the same substituents as those defined above for R₁, preferably by one C₁-C₅ alkyl group.
X is OR₄
wherein R₄ is hydrogen, C₁-C₂₀ alkyl as hereinbefore defined, glycosyl, aralkyl such as benzyl; or C₁-C₆ alkanoyl such as acetyl, propionyl, pivaloyl, stearoyl, or nonadecanoyl or aminoalkanoyl such as aminoacetyl, aminopropionyl and the like or an amino acid residue derived from one of the 20 naturally occurring amino acids viz. alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine and valine, or the optically active isomers thereof, or the racemic mixtures thereof; or R₄ is 1-aminocyclohexylcarbonyl or COOR₁₁ wherein R₁₁ is as hereinbefore defined or R₄ is —(CH₂)ₙ—CHR₁₀—OCOOR₁₁ where R₁₀ and R₁₁ are as hereinbefore defined, or R₄ is C₆H₁₁O₆, PO₂(CH₃)H, PO₃H₂, PO₂(OCH₃)H or SO₃H thus giving respectively the gluconic acid, phosphonic acid, phosphoric acid and sulfonic acid ester derivatives of the compounds;
or X is NR₆R₇, wherein R₆ is H, C₁₋₂₀ alkyl as hereinbefore defined, C₃₋₆ cycloalkyl, aralkyl such as benzyl, phenethyl, or phenylpropyl; C₁₋₂₀ alkanoyl such as COCH₃, COCH₂CH₃, or COC(CH₃)₃, or C₁₋₂₀alkoxycarbonyl such as COOCH₃, COOCH₂CH₃, or COOC(CH₃)₃; aralkyloxycarbonyl such as benzyloxycarbonyl, or amino(C₁₋₂₀) alkanoyl such as aminoacetyl, aminopropionyl and the like, or an amino acid residue derived from one of the 20 naturally occurring amino acids or the optically active isomers thereof, or the racemic mixtures thereof. The amino acid residue is derived from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine. The amino acid residue is derived from a single amino acid or from combinations of amino acids that form dipeptide, tripeptide or polypeptide amino acid unit residues wherein a terminal carboxy group is optionally protected by C₁₋₄ alkyl or aralkyl groups and a terminal amino group is optionally protected by a ^{t}-Boc (teritarybutyloxycarbonyl), F-Moc (fluorenylmethoxycarbonyl) or Cbz (benzyloxycarbonyl) group or R₆ may also be COOR₁₁ wherein R₁₁ is as hereinbefore defined or R₆ is C₆H₁₁O₆ thus giving the gluconic acid ester derivative of the compounds.
R₇ is H, C₁₋₆ alkyl as hereinbefore defined, C₃₋₆ cycloalkyl, aralkyl such as benzyl, phenethyl, or phenylpropyl; C₁₋₆ alkanoyl such as COCH₃, COCH₂CH₃, COC(CH₃)₃, aralkyloxycarbonyl such as benzyloxycarbonyl or amino (C₁₋₂₀)alkanoyl such as aminoacetyl, aminopropionyl, etc.; or an amino acid residue derived from one of the 20 naturally occurring amino acids or the optically active isomers thereof, or the racemic mixtures thereof. The amino acid residue is derived from alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine. The amino acid residue is derived from a single amino acid or from combinations of amino acids that form dipeptide, tripeptide or polypeptide amino acid unit residues, wherein a terminal carboxy group is optionally protected by C₁₋₄ alkyl or aralkyl groups and a terminal amino group is optionally protected by a ^{t}-Boc (teritarybutyloxycarbonyl), F-Moc (fluorenylmethoxycarbonyl) or Cbz (benzyloxycarbonyl) group or
R₇ may be C₆H₁₁O₆ thus giving the gluconic acid ester derivative of the compounds. R₈/R₈′ are substituents at the 3/3-position of the piperidino ring and are the same or different and represent H, C₁₋₆ alkyl, substituted C₁₋₆ alkyl wherein the substituent is amino, hydroxy, halogen such as one or more fluorine, chlorine, or bromine atoms; alkylamino, or aralkyl such as benzyl.
R₉ is a substituent at the 4-position or 5-position of the piperidino ring and represents H, C₁₋₆ alkyl, C₁₋₅ alkylamino, C₁₋₃ dialkylamino or aryl, aralkyl such as benzyl or phenethyl or a trihaloalkyl such as trifluoromethyl.

18. A method or pharmaceutical composition according to claim 12, wherein the antibacterial agent is at least one selected from azithromycin, tigecycline, S-(-)-9-Fluoro-8-(4-hydroxy-piperidin-1-yl)-5-methyl-6,7-dihydro-1-oxo-1H, 5H-benzo [i,j] quinolizine-2-carboxylic acid L-arginine salt tetrahydrate; 2′S, 5S-(-)-9-Fluoro-8-(4-alaninyloxy-piperidin-1-yl)-5-methyl-6,7-dihydro-1-oxo-1H, 5H-benzo [i,j] quinolizine-2-carboxylic acid methane sulfonic acid salt; (RS)-1-Cyclopropyl-6-fluoro-8-methoxy-7-(4-amino-3,3-dimethyl piperidin-1-yl)-1,4-dihydro-4-oxo-quinoline-3-carboxylic acid hydrochloride; (5)-1-Cyclopropyl-6-fluoro-8-methoxy-7-(4-amino-3,3-dimethyl piperidin-1-yl)-1,4-dihydro-4-oxo-quinoline-3-carboxylic acid hydrochloride monohydrate; (RS)-1-Cyclopropyl-6-fluoro-8-methyl-7-(4-amino-3,3-dimethyl piperidin-1-yl)-1,4-dihydro-4-oxo-quinoline-3-carboxylic acid; (S)-1-Cyclopropyl-6-fluoro-8-methyl-7-(4-amino-3,3-dimethyl piperidin-1-yl)-1,4-dihydro-4-oxo-quinoline-3-carboxylic acid hydrochloride; or trans-1-Cyclopropyl-6-fluoro-8-methyl-7-(4-hydroxy-3-methyl piperidin-1-yl)- 1,4-dihydro-4-oxo-quinoline-3-carboxylic acid.

19. A method or a pharmaceutical composition according to claim 13, wherein the antibacterial agent is at least one ansamycin antibacterial agent selected from geldanamycin or herbimycin.

20. A method or a pharmaceutical composition according to claim 13, wherein the antibacterial agent is at least one carbacephem antibacterial agent selected from loracarbef (any other example?)

21. A method or a pharmaceutical composition according to claim 13, wherein the antibacterial agent is at least one carbapenem antibacterial agent selected from ertapenem, doripenem, imipenem, meropenem or sulopenem.

22. A method or a pharmaceutical composition according to claim 13, wherein the antibacterial agent is at least one cephalosporin antibacterial agent selected from cefadroxil, cefazolin, cefalotin, cefalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, ceftobiprole, ceftarolin, or CXA 101.

23. A method or a pharmaceutical composition according to claim 13, wherein the antibacterial agent is at least one glycopeptide antibacterial agent selected from teicoplanin, vancomycin, dalbavancin, telavancin, oritavancin.

24. A method or a pharmaceutical composition according to claim 13, wherein the antibacterial agent is at least one macrolide antibacterial agent selected from azithromycin, clarithromycin, dirithromycin, erythromycin, roxithromycin, troleandomycin, telithromycin or spectinomycin, CEM 101, or EDP 420

25. A method or a pharmaceutical composition according to claim 13, wherein the antibacterial agent is at least one penicillin antibacterial agent selected from amoxicillin, ampicillin, azlocillin, carbenicillin, cloxacillin, dicloxacillin, flucloxacillin, mezlocillin, meticillin, nafcillin, oxacillin, penicillin, piperacillin, ticarcillin or mecillinam.

26. A method or a pharmaceutical composition according to claim 13, wherein the antibacterial agent is at least one polypeptide antibacterial agent selected from bacitracin, colistin or polymyxin-B.

27. A method or a pharmaceutical composition according to claim 13, wherein the antibacterial agent is at least one sulfonamide antibacterial agent selected from mafenide, sulfonamidochrysoidine, sulfacetamide, sulfadiazine, sulfamethizole, sulfanilimide, sulfasalazine, sulfisoxazole, trimethoprim, or trimethoprim-sulfamethoxazole

28. A method or a pharmaceutical composition according to claim 13, wherein the antibacterial agent is at least one tetracycline antibacterial agent selected from demeclocycline, doxycycline, minocycline, oxytetracycline, tetracycline or tigecycline, or amadacycline.

29. A method or a pharmaceutical composition according to claim 12, wherein the antibacterial agent is at least one antibacterial agent selected from arsphenamine, chloramphenico, clindamycin, lincomycin, ethambutol, fosfomycin, fusidic acid, furazolidone, isoniazid, linezolid, metronidazole, mupirocin, nitrofurantoin, platensimycin, pyrazinamide, quinupristin, dalfopristin, rifampicin, thiamphenicol, tinidazole, dapsone, clofazimine, aztreonam, nocardicin, or aztreonam, clavulanic acid, tazobactam, sulbactam or NXL104.

30-36. (canceled)

37. A pharmaceutical composition according to claim 6, further comprising one or more pharmaceutically acceptable carriers.
